(19) United States
(12) Patent Application Publication
(10) Pub. No.: US 2025/0222113 A1
(43) Pub. Date: Jul. 10, 2025

(54) Hybrid Lipid Nanoparticle Comprising an Inorganic Particle and an Agent of Interest

(71) Applicants: The University of British Columbia, Vancouver (CA); Corporation de L'École Polytechnique De Montréal, Montréal (CA)

(72) Inventors: Pieter Cullis, Vancouver (CA); Jayesh Kulkarni, Vancouver (CA); Igor Jigaltsev, Vancouver (CA); Yuen Yi Tam, Richmond (CA); Antoine Uzel, Lyon (FR); Morteza Hasanzadeh Kafshgari, Munich (DE); Michel Meunier, Pierrefonds (CA)

(21) Appl. No.: 18/837,957

(22) PCT Filed: Feb. 14, 2023
(86) PCT No.: PCT/CA2023/050191

§ 371 Date: Aug. 13, 2024

Related U.S. Application Data
(60) Provisional application No. 63/310,065, filed on Feb. 14, 2022.
(60) Provisional application No. 63/340,678, filed on May 11, 2022.

(51) Int. Cl. A61K 47/54; A61K 9/00; A61K 9/51; A61K 41/00; A61K 47/69; A61K 49/00

(52) U.S. Cl. CPC A61K 47/543; A61K 9/0019; A61K 9/5115; A61K 41/0028; A61K 47/6929; A61K 49/00

(57) ABSTRACT

This application relates to lipid nanoparticles, which include: a helper lipid and an ionizable lipid, at least one lipid layer surrounding an interior having at least one aqueous portion, an encapsulated inorganic particle, and an agent of interest (e.g. therapeutic, diagnostic, or theranostic agents). The ionizable lipid is present at between 2 mol % and 30 mol % relative to total lipid. The agent of interest is a hydrophilic agent present in the at least one aqueous portion or is a lipophilic agent present in the at least one lipid layer. This application also relates to methods of making the lipid nanoparticle, as well as methods of using the lipid nanoparticle (e.g. for therapy or diagnostics), including using external stimuli (e.g. laser irradiation) to release the agent of interest.

### PRIORITY

This application claims priority to U.S. Provisional Patent Application No. 63/310,065 filed Feb. 14, 2022 and U.S. Provisional Patent Application No. 63/340,678 filed May 11, 2022, each of which is incorporated by reference in its entirety.

### TECHNICAL FIELD

The disclosure relates to lipid nanoparticles (LNPs), particularly those comprising an inorganic particle and an agent of interest and methods of making same.

### BACKGROUND

Current therapies using small molecular therapeutic agents suffer from the limitation that less than 0.01% of the systemically administered drug is delivered to a target site such as tumour tissue. There have been many efforts to enhance delivery to target tissue while sparing sensitive organs. Examples include implanted (macroscale) timed-release devices that slowly release drug or systemically (i.v.) administered nanocarriers containing drug cargo that preferentially accumulate in a disease site, such as a tumour. Implanted devices suffer from the need for accurate placement in the region of tumours, among other limitations. Systemically administered nanocarriers, on the other hand, require stable drug encapsulation in order to be delivered to the disease site. However, stable encapsulation can prove challenging for drug release at the target site since encapsulated drug has limited bioavailability. While triggered release at a target site in response to external stimuli could address this, targeted release after arrival at a disease site has proven difficult to achieve in practice.

Lipid nanoparticles (LNPs) are well-established nanocarriers for the delivery of a wide range of cargos to a target site in the body (e.g. see: Bangham et al., 1965, J Mol Biol, vol 13, no. 1, pp. 238-52; Allen and Cullis, 2013, Adv Drug Deliv Rev, vol. 65, no. 1, pp. 36-48; Brader et al., 2021, Biophysical Journal, vol. 120, no. 14, pp. 2766-2770). LNP drug delivery systems represent a mature technology for delivery of small molecule drugs (such as anticancer drugs) with nine i.v. injectable LNP drugs that have been approved by regulatory authorities worldwide. A variety of nanoparticles have been tested as potential delivery and imaging agents. Gold nanoparticles (GNP or AuNP) have been identified as promising imaging candidates (Jahangirian et al., Int J Nanomedicine 2019, 14, 1633-1657; and Fernandes et al., J Photochem Photobiol B 2021, 218, 112110 and have potential controlled release properties (Whitener et al., J Biomed Mater Res A 2021, 109 (7), 1256-1265). GNP systems have additional possibilities for causing triggered release of contents as they can “explode” in response to high energy pulsed laser radiation (Letfullin, R. R.; Joenathan, C.; George, T. F.; Zharov, V. P., Laser-induced explosion of gold nanoparticles: potential role for nanophotothermolysis of cancer. Nanomedicine (Lond) 2006, 1 (4), 473-80). However, previously reported hybrid LNP-GNP systems usually lack one or more of the characteristics necessary for clinical utility.

There is a need in the art for nanoparticle formulations containing inorganic particles that provide improvements and/or provide useful alternatives relative to known formulations.

### SUMMARY

Embodiments disclosed herein represent improvements on previous efforts to entrap both inorganic particles and therapeutic and/or imaging agents into lipid nanoparticles (LNPs) and/or provide useful alternatives thereof.

In some embodiments, the disclosure is based, at least in part, on the discovery that LNPs containing ionizable lipid at low levels display a unique morphology that results in the efficient encapsulation of both an agent of interest (e.g., therapeutic agent or diagnostic/imaging agent) and an inorganic particle within the same lipid nanoparticle. In particular, such hybrid LNPs comprise an internal core having an aqueous portion that is capable of loading high levels of the agent, but at the same time such LNPs are capable of accommodating high levels of the inorganic particle therein. The lipid nanoparticles may find use in a broad range of clinical applications relying on the triggered release of LNP contents. In addition, such LNPs comprise lipid components and lamellar structures that may enable long circulation lifetimes to access extrahepatic tissues.

Various embodiments relate to a lipid nanoparticle comprising: a helper lipid and an ionizable lipid, wherein the ionizable lipid is present at between 2 mol % and 30 mol % relative to total lipid; at least one lipid layer surrounding an interior having at least one aqueous portion; an encapsulated inorganic particle; and an agent of interest, wherein the agent of interest is a hydrophilic agent present in the at least one aqueous portion or is a lipophilic agent present in the at least one lipid layer.

Further provided is a method for producing such LNPs that can facilitate high loading efficiency of the therapeutic and/or imaging agent and that is scalable.

Various embodiments relate to a method for producing a lipid nanoparticle entrapping an inorganic particle and an agent of interest, the method comprising: (i) combining in two separate streams a first preparation of lipids dissolved in a solvent and a second preparation of an aqueous solution of an inorganic particle to produce a combined stream, thereby forming in the combined stream a lipid nanoparticle encapsulating the inorganic particle; (ii) introducing a loading medium to an external solution of the lipid nanoparticle thereby formed, the external solution comprising the solvent, and allowing the loading medium to become entrapped in the lipid nanoparticle, thereby producing a lipid nanoparticle comprising the inorganic particle and the entrapped loading medium in an internal compartment thereof; and (iii) introducing the agent of interest to an external solution of the lipid nanoparticle comprising entrapped loading buffer and allowing the agent of interest to be actively loaded into the lipid nanoparticle in response to the entrapped loading medium, thereby producing the lipid nanoparticle entrapping an inorganic particle and the agent of interest.

Various embodiments relate to a method for producing a lipid nanoparticle comprising an inorganic particle core and a lipophilic agent of interest, the method comprising: combining in two separate streams a first preparation of lipids dissolved in a solvent and a second preparation of an aqueous solution of an inorganic particle to produce a combined stream, thereby forming in the combined stream a lipid nanoparticle encapsulating the inorganic particle core; wherein the lipids in the first preparation comprise a helper lipid, an ionizable lipid, and the lipophilic agent of interest; wherein the ionizable lipid is present at between 2 mol % and 30 mol % relative to total lipid, optionally between 5 mol % and 15 mol % relative to total lipid; and wherein the helper lipid is present at a concentration of at least 20 mol %, optionally at least 30 mol %, optionally at least 40 mol %.

Various embodiments relate to use of a lipid nanoparticle disclosed herein for delivering the agent of interest to a subject, wherein the lipid nanoparticle is for administration to the subject followed by administration of a stimulus to a region of the subject, the stimulus causing the inorganic particle in the lipid nanoparticle to cause release of the agent of interest from the lipid nanoparticle.

In some embodiments, this disclosure relates to lipid nanoparticles composed of metal nanoparticles. In certain examples, the lipid nanoparticles comprise two separate internal layers/chambers for respectively encapsulating an agent in the aqueous core and metal nanoparticles in the lipid layer (e.g., bilayer). Taking advantage of pulsed femto- and nano-second lasers for providing a localized energy distribution for few nanoseconds in few nanometers, the formulated liposomal/plasmonic nanocarriers are employed for the site-specific light-triggered delivery of an agent (e.g., a pH gradient loadable drug such as Dox) into a cell in vitro or in vivo.

Various embodiments relate to a lipid nanoparticle comprising: an ionizable lipid content of between 2 mol % and 30 mol %; at least one of a hydrophilic polymer-lipid conjugate and a sterol; a helper lipid content of greater than 30 mol % to form a bilayer surrounding an aqueous portion; an inorganic particle present in the bilayer; and a therapeutic agent and/or imaging agent present in the aqueous portion, wherein the therapeutic agent and/or imaging agent is releasable from the lipid nanoparticle by an irradiation.

Various embodiments relate using the lipid nanoparticles disclosed herein for treating a subject (e.g. mammalian subject) comprising triggered release of the agent at a bodily target site. Various embodiments relate to a method of medical treatment comprising administering the lipid nanoparticle as disclosed herein to a subject (e.g. a mammalian subject) in need of such treatment; and subjecting the lipid nanoparticle to an irradiation to trigger release of the agent at a bodily target site.

Other objects, features and advantages of the present disclosure will be apparent to those of skill in the art from the following detailed description and figures.

### BRIEF DESCRIPTION OF THE FIGURES

FIGS. 1A-F shows the encapsulation of gold nanoparticles into hybrid LNP systems with the lipid composition DODAP/DSPC/Chol/PEG-DSPE, 10/49/40/1 mol/mol) as visualized by cryo-TEM. (A) LNPs formed in absence of GNPs at pH 4. (B) LNPs formed in absence of GNPs at pH 4, and then dialyzed against PBS. (C) LNP-GNPs formed at pH 4, Au/L 2.2×10¹³ particles/mol lipid. Arrows indicate the “dumbbells” where GNPs bridge two vesicles. (D) LNP-GNPs formed at pH 7.4, Au/L 2.2×10¹³ particles/mol lipid, (E) LNP-GNPs formed at pH 7.4, Au/L 6.6×10¹³ particles/mol lipid, (F) LNP-GNPs formed at pH 7.4, Au/L 8.8×10¹³ particles/mol lipid. Size bar represents 100 nm.

FIG. 2 shows normalized absorbance at 520 nm, a.u. vs initial AuNP/Lipid ratios for LNP-GNP formulations with the lipid composition DODAP/DSPC/Chol/PEG-DSPE, 10/49/40/1 mol/mol measured after removal of unentrapped gold (lipid concentrations normalized to 1.25 mg/ml). The inset shows normalized absorbance spectra obtained from LNP-GNP systems after removal of unentrapped gold (Au/L ratios, from top to the bottom: 8.8×10¹³, 6.6×10¹³, 4.4×10¹³, 3.3×10¹³, 2.2×10¹³ and 1.1×10¹³ particles/mol lipid).

FIG. 3 is a bar graph showing AuNP entrapment % as a function of initial AuNP/Lipid ratio for ratios for LNP-GNP formulations with the lipid composition DODAP/DSPC/Chol/PEG-DSPE, 10/49/40/1 mol/mol. The Au/L ratios employed were: (1) 1.1×10¹³ (2) 2.2×10¹³, (3) 3.3×10¹³, (4) 4.4×10¹³, (5) 6.6×10¹³ and (6) 8.8×10¹³ particles/mol lipid.

FIGS. 4A-B are bar graphs showing ammonium sulfate (AS) entrapment within LNP-GNP formulations (DODAP/DSPC/Chol/PEG-DSPE, 10/49/40/1 mol/mol) prepared containing GNP (Au/L ratio 2.2×10¹³ particles/mol) and loaded with AS by adding AS after LNP formation at pH 4, prior to dialysis against PBS to remove ethanol and raise the pH to pH 7.4. The AS concentration in the pH 4 formulation mix was: (1) 300 mM; (2) 450 mM; (3) 600 mM. (A) indicates the entrapped ammonium-to-lipid ratio (mol/mol) and (B) the corresponding ammonium trapping efficiencies following dialysis against PBS.

FIGS. 5A-B shows doxorubicin entrapment (%) vs time for LNP-GNP formulations encapsulating ammonium sulfate. (A) Depicts doxorubicin loading into LNP-GNP systems prepared in the presence of 300 mM (▪), 450 mM (●) and 600 mM (▴) AS at the pH 4 stage of formulation followed by dialysis against PBS (initial drug to lipid ratio 0.1 wt/wt). (B) Shows doxorubicin entrapment at various drug-to-lipid ratios (0.05 wt/wt (▪), 0.1 wt/wt (●) and 0.2 wt/wt (▴)) using LNP-GNP systems prepared in 450 mM AS at the pH 4 stage.

FIGS. 6A-F depict design and characteristics of exemplary LNP/AuNPs/Dox systems. (A) is a Cryo-TEM image of LNP (DODAP/DSPC/Chol/PEG-DSPE, 10/49/40/1 mol/mol) containing AuNP (Au/L 2.2×10¹³ particles/mol) prepared in 450 mM ammonium sulfate (AS) and loaded with doxorubicin at 0.1 (wt/wt) drug-to-lipid ratio. The drug forms a rod-shaped precipitate in the center of the LNP-AuNP pointed by the black arrows. The black dots are the 5 nm AuNP. The LNPs are spherical shape with an average diameter of 100 nm. (B) is a Cryo-TEM imaging of LNP/AuNPs systems; the shape of the LNPs is similar to the one with Dox in the aqueous center. (C) is a schematic representation of the different LNP samples. Dox is in the aqueous center while gold nanoparticles are placed in the outer layer of the LNPs. (D) is a table showing physicochemical properties of LNPs, hydrodynamic diameter, size distribution (PDI) and zeta potential of control LNPs, and LNPs with and without AuNP and Dox (measured at the same concentration of Dox of 0.1 mg/mL). (E) shows fluorescence spectra of LNPs/AuNPs/Dox (bottom spectrum) and LNPs/Dox (top spectrum) between 570 nm and 610 when an excitation of 505-555 nm is used. Fluorescence is due to Dox, which is naturally fluorescent. (F) shows absorbance spectra of LNP/AuNP/Dox (top at maximum), LNP/Dox (2^{nd} from top at maximum), LNP/AuNP (2^{nd} from bottom at maximum), LNP (bottom at maximum) and Dox (middle at maximum). Spectra were measured at the same concentration of Dox or corresponding concentration of LNPs.

FIGS. 7A-C. Dox release following irradiation with nanosecond laser with 3 different protocols. FIG. 7A shows fluorescence images of MDA-MB-231 cells treated with control LNPs (without AuNPs) and with AuNPs, with and without irradiation. Concentration of 50 μg/mL of LNPs. Cell membrane staining with Calcein AM in green and Dox-related red fluorescence. Irradiation with an objective of 4× magnification, numerical aperture of NA=0.13 performed at energy of 14.2 μJ, scanning speed of 50 μm/s, and scanning lines separated by 5 μm step. The pulse width is 7.7 nanosecond. The protocol to perform these irradiations is schematized below the images. Irradiation was performed before the washing phase, occurring 4 hours after the addition of nanoparticles to the cells. The image phase was done 20 hours later. The exact same protocols were followed for the control samples (i.e no irradiation but same incubation time, same waiting time before taking picture and washing step). FIG. 7B shows fluorescence images of MDA-MB-231 cells with incubation of LNPs/AuNPs/Dox for one hour at a concentration of 50 μg/mL of LNPs. Irradiation performed: after washing step in image 1, just before washing step in image 2 and no irradiation in image 3. Irradiation was performed at an energy of 19.7 μJ, 50 μm/s and 5 μm step. The exact protocol followed is detailed under the 3 images. Two irradiations were done on 2 different areas, one before and one after the washing step. FIG. 7C shows fluorescence images of MDA-MB-231 cells treated with control LNPs (without AuNPs) and with AuNPs, with and without irradiation. Concentration of 50 μg/mL of LNPs. Irradiation was performed at an energy of 19.7 μJ, 50 μm/s, and 5 μm step. Calcein AM staining of the cell membrane in green and Dox-related red fluorescence. The protocol schematized below the images corresponds to irradiation occurring after cell washing. LNPs were in solution with the cells for only 15 minutes.

FIG. 8 shows the normalized fluorescent intensities of LNPs containing Dox with or without gold nanoparticles (AuNPs) after irradiation with nanosecond laser at different energies (from 0 to 19.7 μJ) with 2 different scan parameters namely speed 1: 100 μm/s and 10 μm step and speed 2: 50 μm/s and 5 μm step. The normalized value was fixed to 1 for non-irradiated sample with LNP/Dox. Incubation of cells was for 4 hours with LNP/Dox and LNP/AuNP/Dox at a concentration of 50 μg/mL.

FIGS. 9A-C show Dox release following irradiation with femtosecond laser (wavelength 800 nm, pulse width 55 fs, 1 kHz repetition rate, 35 am spot diameter). FIG. 9A shows fluorescence images of MDA-MB-231 cells treated with control LNPs (without AuNPs) and with AuNPs, with and without irradiation. The irradiation protocol is schematized below the images. Incubation of cells used a concentration of 50 μg/mL LNP/Dox or LNP/AuNP/Dox. FIG. 9B shows normalized fluorescent intensities of LNPs containing Dox after irradiation with femtosecond laser at different fluences (from 0 to 73 mJ/cm²) with 2 different scan parameters namely speed 1: 3 cm/s and 30 μm step and speed 2: 6 cm/s and 60 μm step. The normalized value was fixed to 1 for non-irradiated sample with LNP/Dox. FIG. 9C shows a schematic representation of irradiation space for each speed. Dots represent the laser pulses, and the arrows represent the laser path.

### DETAILED DESCRIPTION

### Lipid Nanoparticle

The lipid nanoparticle (LNP) described herein comprises a helper lipid and a cationic lipid at levels selected to produce an LNP having a morphology that is particularly amenable to efficient encapsulation of both an inorganic particle (e.g., metal nanoparticle) and an agent (e.g. a therapeutic or diagnostic agent), such as those that are loadable by active loading methods. The helper lipid may be included at greater than 30 mol %. In another embodiment, the cationic lipid is present at between 2 mol % and 30 mol % relative to total lipid. Such LNPs are particularly well-suited for the triggered release of the LNP contents in therapeutic or diagnostic applications.

In one non-limiting example, a lipid layer, such as a bilayer or other lamellar structure, surrounds the interior of the LNP, which interior comprises an aqueous portion. As used herein, the “interior” (also referred to as “interior core” or just “core”) of a LNP refers to everything inside the outermost lipid layer (e.g. outermost bilayer) separating the lipid nanoparticle from its external environment. It has been observed that as the proportion of ionizable lipid is decreased further, the size of the hydrophobic core may decrease and the number of lamellae decreases. Without being limiting, LNPs incorporating inorganic particles as described herein, such as at their maximum encapsulation levels, may contain essentially no hydrophobic core within the internal core or a small region thereof. In such embodiments, the inorganic particle may be at least partially complexed to the ionizable lipid. Without being limiting, the inorganic particles may be located at an intersection of a lipid layer or layers (e.g., lamellae). Advantageously, the aqueous portion in such lipid nanoparticles may accommodate a therapeutic agent and/or a diagnostic agent that has been actively loaded at high encapsulation efficiency.

The lipid nanoparticle typically has a mean diameter of between 50 and 180 nm, 60 and 150 nm or 65 and 130 nm or any range therebetween. The lipid nanoparticle may be elongate or circular in cross-section.

### Helper Lipid

In the context of the present disclosure, the term “helper lipid” includes any vesicle-forming or liposome-forming lipid. Helper lipids therefore include amphipathic lipids (e.g. alkyl chains of C14-C18 with 0-3 double bonds) in which the polar (i.e. hydrophilic) region contains phosphate, carboxyl, sulfate, sulfonyl, amino or nitro groups. In some embodiments, the helper lipids are phospholipids (e.g. phosphatidylcholine, sphingomyelin, and the like, or mixtures thereof). The helper lipid may be cationic, anionic, or zwitterionic at physiological pH (e.g. pH ˜7.0), and may be net negatively charged, net positively charged, or have net neutral charge. In some embodiments, the helper lipid has net neutral charge. Non-limiting examples of helper lipids include DLPC (12:0), DMPC (14:0), DPPC (16:0), DSPC (18:0), DOPC (18:1), DMPE (14:0), DPPE (16:0), DOPE (18:1), DMPA (14:0), DPPA (16:0), DOPA (18:1), DMPG (14:0), DPPG (16:0), DOPG (18:1), DMPS (14:0), DPPS (18:1), DOPS (18:1), DPOE-glutary (14:0), tetramyristoyl cardiolipin (14:0), DOTAP (18:1), and in some embodiments the helper lipid is one or a mixture of two or more of the foregoing. In some embodiments, the helper lipid is DLPC (12:0), DMPC (14:0), DPPC (16:0), DSPC (18:0), DOPC (18:1), DMPE (14:0), DPPE (16:0), DOPE (18:1), or a combination of two or more thereof. In some embodiments, the helper lipid is selected from sphingomyelin, distearoylphosphatidylcholine (DSPC), dioleoylphosphatidylcholine (DOPC), 1-palmitoyl-2-oleoyl-phosphatidylcholine (POPC), dipalmitoyl-phosphatidylcholine (DPPC), or mixtures of two or more thereof. In certain embodiments, the helper lipid is DOPC, DSPC, sphingomyelin, or mixtures of two or more thereof. In one embodiment, the helper lipid is DSPC. The helper lipid content may be a single helper lipids or mixtures of two or more types of different helper lipids.

The helper lipid content in some embodiments is greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of helper lipid content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the total helper lipid content is from 20 mol % to 70 mol % or 25 mol % to 70 mol % or 30 mol % to 70 mol % or 35 mol % to 70 mol % or 40 mol % to 70 mol % of total lipid present in the lipid nanoparticle.

The phosphatidylcholine content of the lipid nanoparticle in some embodiments is greater than 15 mol %, greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of phosphatidylcholine content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the phosphatidylcholine content is from 20 mol % to 60 mol % or 25 mol % to 60 mol % or 30 mol % to 60 mol % or 35 mol % to 60 mol % or 40 mol % to 60 mol % of total lipid present in the lipid nanoparticle. The phosphatidylcholine lipid content is determined based on the total amount of lipid in the lipid nanoparticle, including the sterol.

### Ionizable Lipid

The term “ionizable lipid” refers to any of a number of lipid species that carry a net positive or negative charge at a selected pH, such as physiological pH (e.g., pH of about 7.0). The ionizable lipid may be cationic, anionic, or zwitterionic.

In some embodiments, the ionizable lipid(s) comprise a cationic lipid and in certain embodiments has a head group comprising an amino group. In select embodiments, the cationic lipids comprise a protonatable tertiary amine (e.g., pH titratable) head group, C14 to C18 alkyl chains, linker regions between the head group and alkyl chains, and 0 to 3 double bonds. Non-limiting examples of cationic ionizable include 1,2-dioleoyl-3-dimethylammonium propane (DODAP), 1,2-dioleyloxy-3-dimethylaminopropane (DODMA). Such lipids include, but are not limited to DLin-KC2-CMA (KC2), DLin-MC3-DMA (MC3), 1,2-dioleoyl-3-dimethylammonium propane (DODAP), 1,2-dioleyloxy-3-dimethylaminopropane (DODMA), N,N-dioleyl-N,N-dimethylammonium chloride (DODAC), 1,2-dioleoyl-3-(trimethylammonium) propane (DOTAP), and 1,2-di-O-octadecenyl-3-trimethylammonium propane (DOTMA). In some embodiments the ionizable lipid is one or a mixture of two or more cationic lipids, e.g. two or more of those disclosed herein.

In some embodiments, the ionizable lipid(s) comprise an anionic lipid. In select embodiments, the anionic lipids comprise an anionic head group (e.g. phosphate, carboxyl, sulfate, sulfonyl, nitro, and the like), C14 to C18 alkyl chains, linker regions between the head group and alkyl chains, and 0 to 3 double bonds, and optionally pegylated (PEG attached to the head group). Non-limiting examples of anioinic ionizable lipids include DMPA (14:0), DPPA (16:0), DOPA (18:1), DMPG (14:0), DPPG (16:0), DOPG (18:1), DMPS (14:0), DPPS (18:1), DOPS (18:1), DPOE-glutaryl (14:0), tetramyristoyl cardiolipin (14:0). In certain embodiments, a mixture of ionizable lipids is included in the lipid nanoparticle.

In some embodiments, the ionizable lipids comprise one or more charged lipids as described in WO 2021/026647, the entirety of which is incorporated herein by reference. In some such embodiments, the charged lipid(s) is a lipid(s) comprising a branched lipid moiety L having the structure of Formula I (with definitions of terms incorporated by reference from WO 2021/026647).

Formula I:

A-(V)ₘ-Z-L (I)
A is a head group that is ionizable, permanently charged or zwitterionic;
(V)ₘ is an optional —(CR1R2)ₘ—, and m is 1 to 10 or 2 to 6, wherein R1 and R2 are each independently: hydrogen, optionally substituted alkyl, alkenyl, alkynyl, aryl, cycloalkyl, cycloalkylalkyl, or a heterocycle; and
Z-L has a structure of Formula II, IIa or IIb below,
and wherein L is a hydrocarbon structure and has a moiety of Formula IIIc below.

### Formula IIIc:

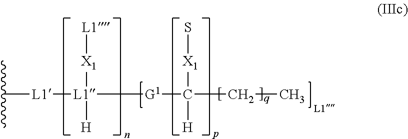

wherein a scaffold carbon chain of L is denoted by L1′-L1″-G¹-CH—[CH₂]_{q}—CH₃, and
wherein the total number of carbon atoms in the L carbon backbone is 10 to 30;
L1′ is a linear hydrocarbon chain having no heteroatoms and has 5-12 (or more) carbon atoms and 0-3 cis or trans double bonds;
L1″ is a carbon atom;
each X1 is independently selected from an ether, ester and carbamate group;
L1′″ is a carbon backbone portion of the scaffold carbon chain L and is depicted by G¹-CH—[CH₂]_{q}—CH₃ and wherein G¹ is a hydrocarbon chain of 0-4 carbon atoms, optionally having one cis or trans double bond;
wherein n is 0 to 4;
wherein p is 1 to 4;
wherein n+p is 1 to 4;
q is 0 to 20;
wherein each S and L1″″ is a hydrocarbon side chain and is independently:
   (a) a linear or branched terminating hydrocarbon chain with 0 to 5 cis or trans C═C and 2 to 30 carbon atoms and conjugated to one of a respective X1 at any carbon atom in its hydrocarbon chain thereof; or
   (b) a branched hydrocarbon structure of Formula IIIc,
wherein each one of the L1″″ and S hydrocarbon chains in the lipid moiety is optionally substituted with a heteroatom, with the proviso that no more than 2 heteroatoms are substituted in the hydrocarbon chains.

### Formula II Linear Linker Structure:

X1-L_{b},
wherein X1 is optional and X1 is selected from an ether, ester and carbamate group; and
L_{b} is a branched lipid of Formula IIIc.

### Formula IIa Branched Linker Structure:

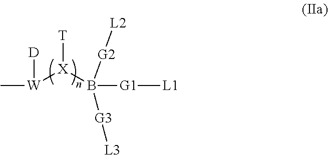

W is optional;
W, if present, is an X1 linkage, N—C(O), N—C(O)O, or N—OC(O);
wherein W is optionally substituted with D, which is an optionally substituted alkyl, alkenyl, alkynyl, aryl, cycloalkyl, cycloalkylalkyl, or heterocycle;
each occurrence of (X)ₙ is an independently selected —(CR1R2)ₙ—; n of (X)ₙ is 0 to 10; and
T is optional and is an alkyl, alkenyl, alkynyl, aryl, cycloalkyl, cycloalkylalkyl, or heterocycle and wherein T is optionally substituted;
B is a carbon atom linked to L1 and L2 via respective G1 and G2;
wherein G1 and G2 are independently selected from an X1;
wherein each of G1 and G2 is independently optionally covalently bonded to B via an intervening (G)ᵤ group as B-(G)ᵤ-G1 or B-(G)ᵤ-G2, respectively;
wherein (G)ᵤ is an independently selected —(CR1R2)ᵤ- wherein R1 and R2 are each independently: hydrogen, optionally substituted alkyl, alkenyl, alkynyl, aryl, cycloalkyl, cycloalkylalkyl, or heterocycle and u is 0 to 16;
wherein G3 is optional and is selected from X1 and optionally covalently bonded to the B via an intervening (G)ᵤ group as B-(G)ᵤ-G3;
L1 is a branched hydrocarbon of Formula IIIc;
L2 is a hydrocarbon chain having 1 to 20 carbon atoms and 0 to 2 cis or trans double bonds or has the structure of Formula IIIc;
L3 if present is hydrogen, a linear or branched hydrocarbon chain having 1 to 20 carbon atoms and 0 to 2 cis or trans double bonds or has the structure of Formula IIIc.

### Formula IIb Ring Structure:

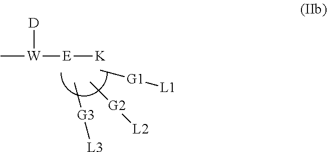

wherein the curved line represents a ring and E and K depict atoms that partially form the structure of the ring, which ring is a substituted or unsubstituted ring having 3 to 8 ring atoms;
wherein at least one of L1, L2 and L3 are bonded to a single atom in the ring, optionally via a respective G1, G2 and G3, wherein each of G1, G2 and G3 is independently optionally covalently bonded to a respective one of the L1, L2 and L3 via an intervening (G)ᵤ, as G1-(G)ᵤ-L1, G2--(G)ᵤ-L2 or G3-(G)ᵤ-L3, respectively;
wherein L1 and optionally L2 and/or L3 of Formula IIb have the structure of Formula IIIc; and
wherein the A moiety is optionally selected from one of:
   (i) ionizable cationic moieties selected from the group consisting of:

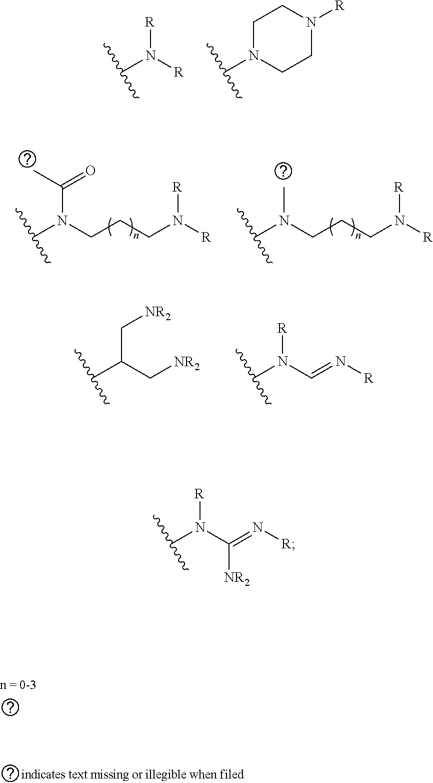

(ii) permanently charged moieties selected from the group consisting of:

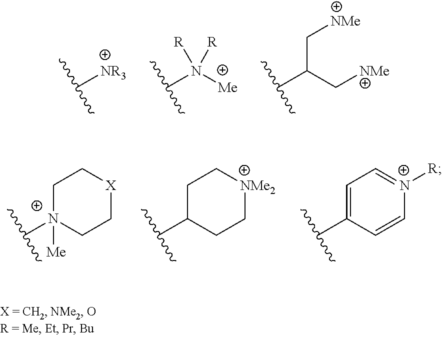

(iii) ionizable anionic moieties selected from the group consisting of:

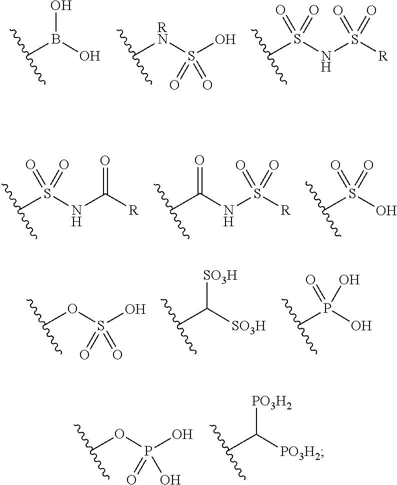

or
(iv) zwitterionic moieties selected from the group consisting of:

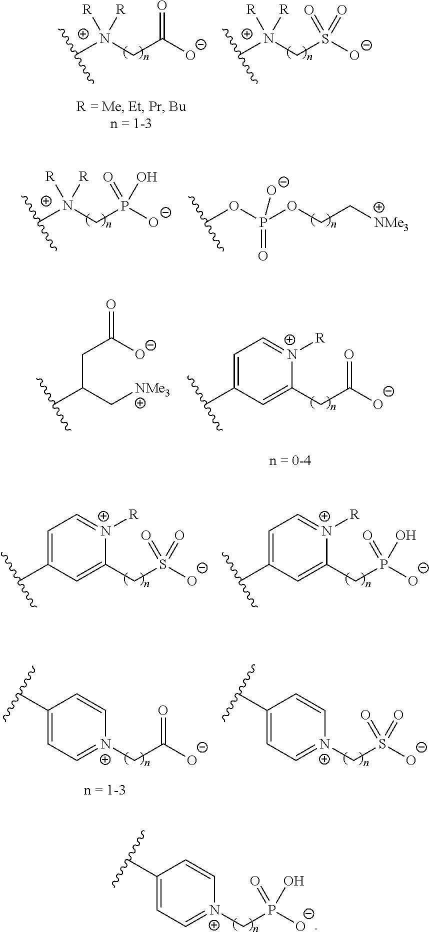

In some embodiments, Z-L has the structure of Formula II; wherein L1′ of Formula IIIc has 5 to 9 carbon atoms and has 0 to 2 cis or trans double bonds; wherein G1 of Formula IIIc is absent, CH₂ or CH₂CH═CH, and wherein the double bond is cis or trans; wherein L1″″, if present, and S of Formula IIIc are independently selected from a hydrocarbon with 0-5 cis or trans CH═CH and 2 to 18 carbon atoms; and wherein q is 1 to 9.

In some embodiments, (V)ₘ is (CH₂)ₘ, wherein m is 1 to 20; Z-L has the structure of Formula IIa; wherein W is an ether, ester or carbamate group and D is absent, and (X)ₙ is (CH₂)ₙ, wherein n is 1 to 10; wherein G1 and G2 are present and are covalently bonded to the B via a (G)ᵤ, as B-(G)ᵤ-L1 or B-(G)ᵤ-L2, wherein (G)ᵤ is (CH₂)ᵤ; wherein G3-L3 is present and is a hydrocarbon selected from CH₃ and CH₂CH₃; or wherein G3-L3 is CH₂X1L3 and L3 is a linear or branched hydrocarbon chain having 1 to 20 carbon atoms and 0 to 2 cis or trans double bonds or has the structure of Formula IIIc.

In some embodiments, Z-L has the structure of Formula IIb, wherein the curved line represents a ring and E and K depict atoms that partially form the structure of the ring, which ring is a substituted or unsubstituted carbon ring having 3 to 6 ring atoms. In select embodiments, the ring comprises 3 or 5 carbon atoms. In select embodiments, at least L1 and L2 are present and are attached to the ring via respective G1 and G2 groups and wherein each G1 and G2 group is optionally covalently bonded to an atom of the ring via an intervening (G)ᵤ, wherein (G)ᵤ is (CH₂)ᵤ and u is 0 to 10 or 0 to 6.

In some embodiments, the R1 or R2 of (V)ₘ is the cycloalkyl that is an optionally substituted mono-, bi-, or tri-cyclic carbon ring.

In some embodiments, the R1 or R2 of (V)ₘ are each independently selected from the heteroatom ring having 4 to 12 ring atoms.

The ionizable lipid content may be less than 30 mol %, less than 25 mol %, less than 20 mol %, less than 18 mol %, less than 15 mol %, less than 12 mol %, less than 10 mol % or less than 5 mol %. In certain embodiments, the ionizable lipid content is from 2 mol % to 30 mol % or 5 mol % to 25 mol % or 7 mol % to 20 mol % of total lipid present in the lipid nanoparticle. In some embodiments, the ionizable lipid is cationic at physiological pH. In one embodiment, the amine to phosphate charge ratio (N/P) of the lipid nanoparticle is between 3 and 15, between 5 and 10, between 6 and 9, between 8 and 10 or between 5 and 8.

### Sterol

The lipid nanoparticle optionally includes a sterol. Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol and the like. In one embodiment, the sterol is present at from 15 mol % to 65 mol %, 18 mol % to 50 mol %, 20 mol % to 50 mol %, 25 mol % to 50 mol % or 30 mol % to 50 mol % based on the total lipid present in the lipid nanoparticle. In another embodiment, the sterol is cholesterol and is present at from 15 mol % to 65 mol %, 18 mol % to 50 mol %, 20 mol % to 50 mol %, 25 mol % to 50 mol % or 30 mol % to 50 mol % based on the total lipid and sterol present in the lipid nanoparticle. In one embodiment, the combined (i) sterol content (e.g., cholesterol or cholesterol derivative thereof); and (ii) helper phospholipid content (e.g., phosphatidylcholine or sphingomyelin) is at least 50 mol %; at least 55 mol %, at least 60 mol %, at least 65 mol %, at least 70 mol %, at least 75 mol %, at least 80 mol % or at least 85 mol % based on the total lipid present in the lipid nanoparticle.

### Hydrophilic Polymer-Lipid Conjugate

In one embodiment, the lipid nanoparticle comprises a hydrophilic-polymer lipid conjugate capable of incorporation into the lipid nanoparticle. The conjugate includes a vesicle-forming lipid having a polar head group, and (ii) covalently attached to the head group, a polymer chain that is hydrophilic. Example of hydrophilic polymers include polyethyleneglycol (PEG) (Nunes et al., 2019, Drug Deliv Transl Res, vol. 9, no. 1, pp. 123-130), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic-polymer lipid conjugate is a PEG-lipid conjugate. Non-limiting examples of PEG-lipid conjugates include DMPE-mPEG-2000 (14:0), DMPE-mPEG-5000 (14:0), DSPE-mPEG-2000 (18:0), DSPE-mPEG-5000 (18:0), DSPE-maleimide PEG-2000 (18:0), DMG-PEG-2000 (14:0), DSG-PEG-2000 (18:0), and the like.

The hydrophilic polymer lipid conjugate may be present in the nanoparticle at 0 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid. In another embodiment, the PEG-lipid conjugate is present in the nanoparticle at 0 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

### Inorganic Particle

The term “inorganic particle” means a nanosize particle that is suitable for formulation in a lipid nanoparticle as described herein and that comprises a suitable metal. The inorganic particle has suitable properties for triggering drug release upon application of a suitable energy wavelength. For example, the inorganic particle may be magnetic. The metal in some non-limiting examples is most advantageously biocompatible and nontoxic. The metal includes but is not limited to gold, silver, iron, copper, nickel, cobalt, platinum, iridium, alloy of two or more thereof, or mixtures thereof. The metal may be present in any form, such as a salt (e.g., oxides, hydroxides, sulfides, phosphates, fluorides or chlorides) or complexed. In some embodiments, the metal is gold or silver. In another embodiment, the inorganic particle comprises a hybrid gold-iron oxide.

The inorganic particle includes without limitation metal nanoparticles, nanoshells, nanocages, quantum dots or upconverting nanoparticles. In some embodiments, the inorganic particle will be in the shape of a sphere or a rod or a nanostar, most typically a sphere. The inorganic particle is typically small and less than 20 nm in diameter, less than 15 nm, less than 10 nm or less than 5 nm in diameter.

In the case of a metal nanoparticle, the metal may be associated with a ligand, such as a “capping agent”. Without being limiting, the capping agent may control the growth, agglomeration, and/or physico-chemical characteristics of the metal nanoparticle. The capping agent may in some embodiments reduce or block reactivity at the periphery of the metal nanoparticle. A capping agent may in some embodiment function as a reducing agent and a capping agent.

In certain advantageous embodiments, the capping agent imparts a negative charge to the metal nanoparticle. If the ionizable lipid is cationic at a desired pH (e.g., physiological or below physiological), the use of a negatively charged capping ligand may facilitate incorporation of the metal nanoparticle into the lipid nanoparticle. In some embodiments, the opposing charges between the ionizable lipid and the metal nanoparticle allow an association or complex to be formed between the negatively charged metal nanoparticle and the positively charged lipid, thereby improving encapsulation efficiency. In one embodiment, the capping agent is a macromolecule, such as but not limited to tannic acid or comprises a citrate ion.

The capping agent in some alternative embodiments may be an amphiphilic molecule comprising a polar head group and a non-polar hydrocarbon tail. Owing to the amphiphilic nature of capping agents, in some embodiments they provide functionality and/or enhance the compatibility with another phase. In one non-limiting example, a non-polar tail interacts with the external medium while the polar head interacts with the metal atom of the nanoparticle.

### Loading an Agent of Interest

In some embodiments, the LNP comprises aqueous soluble loadable agent(s) of interest, e.g. therapeutic, diagnostic, and/or theranostic agent(s), present in the aqueous portion of the LNP. In other embodiments, the LNP comprises hydrophobic or lipophilic loadable agent(s) of interest, e.g. therapeutic, diagnostic, and/or theranostic agent(s), or prodrugs thereof, present in the lipid portion of the LNP. The loadable agents of interest may be any molecule of interest, e.g. small molecules (e.g. small molecule drugs, imaging agents, and the like), proteins (e.g. antibodies and the like), peptides, nucleic acids (e.g. siRNA and the like).

In one embodiment, the agent incorporated into the aqueous portion of the lipid nanoparticle is capable of being actively loaded therein. Agents that may be loaded using pH gradient loading comprise one or more ionizable moieties such that the neutral form of the ionizable moiety allows the drug to cross the lipid nanoparticle membrane and conversion of the moiety to a charged form causes the agent to remain encapsulated within the liposome. Ionizable moieties may comprise amine, carboxylic acid, hydroxyl groups, or any other charged moiety. Agents that load in response to an acidic interior may comprise ionizable moieties that are charged in response to an acidic environment, whereas agents that load in response to a basic interior comprise moieties that are charged in response to a basic environment. In the case of a basic interior, ionizable moieties including but not limited to carboxylic acid or hydroxyl groups may be utilized. In the case of an acidic interior, ionizable moieties including but not limited to primary, secondary and tertiary amine groups may be used. In some embodiments, agents to be loaded into a basic interior using pH gradient loading should be a weak acid or have a pKa ˜2-6 and a molecular weight <1500 g/mol, whereas agents to be loaded into an acidic interior using pH gradient loading should be a weak base or have a pKa ˜6-9 and a molecular weight <1500 g/mol.

Without intending to be limiting, the pH gradient loadable agent may be an anti-neoplastic agent, antimicrobial agent or an anti-viral agent. Non-limiting examples of therapeutic agents that can be loaded into lipid nanoparticles by the pH gradient loading method and therefore may be used in practice of this disclosure include, but are not limited to anthracycline antibiotics such as doxorubicin, daunorubicin, mitoxantrone, epirubicin, aclarubicin and idarubicin; anti-neoplastic antibiotics such as mitomycin, bleomycin and dactinomycin; vinca alkaloids such as vinblastine, vincristine and navelbine; purine derivatives such as 6-mercaptopurine and 6-thioguanine; purine and pyrimidine derivatives such as 5-fluorouracil; camptothecins such as topotecan, irinotecan, lurtotecan, 9-aminocamptothecin, 9-nitrocamptothecin and 10-hydroxycamptothecin; cytarabines such as cytosine arabinoside; antimicrobial agents such as ciprofloxacin and salts thereof.

In some embodiment, the loadable agent of interest is a diagnostic or imaging agent (e.g. contrast agents, such as radiolabelled agents or MRI contrast agents, fluorescent probes, and the like). Such agents may be incorporated in the aqueous portion of the lipid nanoparticle or in the lipid portion of the lipid nanoparticle. For example, but without limitation, soluble contrast agent(s) may be incorporated into the aqueous portion of the LNP.

Hydrophobic agents of interest can be easily loaded in LNP systems by simply mixing them with the lipid components (e.g. see Example 4).

Hydrophilic agents may be converted to a hydrophobic agent, and therefore lipid-loadable, using known methods, including without limitation conjugating a lipid moiety, e.g. as described in WO/2020/191477, which is incorporated by reference in its entirety. Lipid moieties may be conjugated using various linkers, e.g. succinate, ester, amide, hydrazone, ether, carbamate, carbonate, phosphodiester, and the like. Lipid-conjugated agents may be a therapeutic agent, diagnostic agent, a theranostic agent, or any other agent of interest. The linker between the lipid moiety and the agent of interest may be cleaved in vivo (e.g. by an enzyme, pH, and the like). Lipid-conjugated agents may be a prodrug, such that its release/cleavage from the lipid moiety is activated to a therapeutic, or theranostic form, or may be released as a prodrug and is subsequently converted (e.g. biochemically) to its active form. Suitable lipids and linkers (e.g. cleavable and non-cleavable linkers) are known, e.g. as described in WO/2020/191477.

### Nanoparticle Preparation

The hybrid lipid nanoparticles can be prepared using a variety of suitable methods, such as a rapid mixing/solvent (e.g., ethanol) dilution process. Non-limiting examples of preparation methods are described in Jeffs, L. B., et al., Pharm Res, 2005, 22(3):362-72; and Leung, A. K., et al., The Journal of Physical Chemistry. C, Nanomaterials and Interfaces, 2012, 116(34): 18440-18450, each of which is incorporated herein by reference in its entirety.

In order to incorporate an agent into the aqueous portion of the lipid nanoparticle, a loading buffer should be introduced therein to drive uptake of the agent. There are a number of possible ways to introduce loading buffer into the LNP. One possible method involves incorporating the loading buffer into the aqueous medium containing the inorganic particle during a mixing stage with the lipid in the solvent (e.g., ethanol). However, aqueous dispersions of colloidal metal, such as gold, may be sensitive to ionic strength. It is possible in some embodiments that precipitation of the LNP may occur upon introduction of a buffering agent during the mixing stage. To address the possibility of such LNP precipitation upon introduction of a loading solution during mixing, the loading solution may be added to the LNP comprising encapsulated metal subsequent to its formation, followed by uptake of the loading solution into the lipid nanoparticle.

In such embodiments, the method comprises entrapping the inorganic particle in the lipid nanoparticle to produce a lipid nanoparticle comprising entrapped inorganic particle (e.g. metal nanoparticle). This involves combining, in two separate streams, a first preparation of lipids dissolved in a solvent (e.g., ethanol or other suitable solvent) and a second preparation of an aqueous solution of an inorganic particle. The two streams are combined in a suitable mixing device to produce a combined stream, thereby forming the lipid nanoparticle entrapping the inorganic particle. The loading buffer is subsequently added to an external solution of the lipid nanoparticle thereby formed. Since the external solution comprises the solvent (e.g., ethanol or other suitable solvent) used to form the LNP comprising the inorganic particle, and such solvent can facilitate incorporation of the loading buffer into the aqueous portion of the lipid nanoparticle by enhancing the permeability of the LNP lipid layers, this allows the loading buffer to become entrapped in the lipid nanoparticle, thereby producing a lipid nanoparticle comprising the inorganic particle and the entrapped loading buffer in an internal portion or compartment thereof. To create a transmembrane chemical gradient, the original external medium of the lipid nanoparticle is replaced by a new external medium having a different concentration of the species that drives the loading (e.g., protons). The method subsequently comprises introducing the actively loadable agent to an external solution of the lipid nanoparticle comprising entrapped loading buffer and allowing the agent to be actively loaded into the lipid nanoparticle, thereby producing the lipid nanoparticle entrapping both the inorganic particle and the actively loadable agent.

The replacement of the external medium can be accomplished by various techniques, such as, by passing the lipid nanoparticle through a gel filtration column, e.g., a Sephadex column, which has been equilibrated with the new medium (as set forth in the examples below), or by centrifugation, dialysis, or related techniques. For pH gradient loading, the internal medium may be either acidic or basic with respect to the external medium. In such embodiments, after establishment of a pH gradient, a pH gradient loadable agent is added to the mixture and encapsulation of the agent in the lipid nanoparticle occurs as described above.

Loading using a pH gradient may be carried out according to known methods, e.g. as described in U.S. Pat. Nos. 5,616,341, 5,736,155 and 5,785,987, each incorporated herein by reference.

While pH gradient loading is described, the active loading involves the use of any suitable transmembrane chemical gradient across the LNP membrane to induce uptake of an actively loadable agent after the LNP has been formed. This can involve a gradient of one or more ions including Na⁺, K⁺, H⁺, and/or a protonated nitrogen moiety. In other words, active loading techniques that may be used in accordance with this disclosure include, without limitation, pH gradient loading, charge attraction, and drug shuttling by an agent that can bind to the drug.

### Nanoparticle Morphology

The lipid nanoparticles comprise a core that encapsulates both an inorganic particle and an agent that in some embodiments is a therapeutic agent or an imaging agent. By the term “core” or “internal core”, it is meant a trapped or at least partially enclosed volume of the lipid nanoparticle that comprises an aqueous portion and optionally an electron dense region (e.g., hydrophobic core). The aqueous portion and electron dense region, if present, can be visualized by cryo-EM microscopy. In one embodiment, at least about one quarter of the core contains the aqueous portion, or at least about one third of the core contains the aqueous portion, or at least one about one half of the core contains the aqueous portion as determined qualitatively by cryo-EM or other suitable technique. In one embodiment, the shape of the lipid nanoparticle is circular in cross-section or elongate.

The unique morphology may be dependent on the proportion of “bilayer” lipids (helper lipid) in the lipid nanoparticle. It has been observed that as helper lipid (e.g., DSPC) is increased, the helper lipid first forms a monolayer around a core region that is hydrophobic, with subsequent formation of a bilayer surrounding the core. As the proportion of ionizable lipid is decreased further, the size of the hydrophobic region decreases and the number of lamellae increases. Without intending to be limited by theory, it may be proposed that LNPs containing negatively charged inorganic particles at maximum inorganic particle (e.g., gold nanoparticles (GNP)) entrapment levels represent a limiting situation where there is essentially no hydrophobic region in the core and the inorganic particle is complexed or associated with the ionizable lipid and located at the intersection of the lamellae.

In one embodiment, the lipid nanoparticle surface is substantially uncharged as determined by measuring a zeta potential of the LNP as described herein. This may result from an outer lipid layer (e.g., a bilayer) possessing low levels of ionizable lipid and high helper lipid content. In an alternative embodiment, the LNP is unilamellar or multi-lamellar.

Without wishing to be bound by theory, the inorganic particle may be associated or complexed with the ionizable lipid. The encapsulated inorganic particle in some embodiments is present in the lipid nanoparticle in a region of the particle where two lipid layers meet as detected by cryo-TEM microscopy. However, it will be understood that the invention is not constrained by the location or the nature of the incorporation of the inorganic particle within the lipid nanoparticle. That is, the term “encapsulated” is not meant to be limited to any specific interaction between the inorganic particle and the lipid nanoparticle. The inorganic particle may be incorporated in the aqueous portion, within any lipid layer or both.

### Pharmaceutical Formulations

In some embodiments, the disclosure provides a method of treating or imaging cells in a subject by administering at least one lipid nanoparticle to the cells in vivo. The method for treating or imaging cells may further include the application of an external energy source, such as a light source, a laser (continuous wave (cw) or pulsed), x-ray or gamma ray. The energy source will cause at least partial release of the contents of the lipid nanoparticle to enable an imaging and/or therapeutic effect. As is known to those of skill in the art, irradiating the lipid nanoparticle with a suitable energy source increases the degradation rate of the lipid nanoparticle.

In some embodiments, the lipid nanoparticle of the disclosure is part of a pharmaceutical composition and is administered to treat and/or prevent a disease condition. The treatment may provide a prophylactic (preventive), ameliorative or a therapeutic benefit. The pharmaceutical composition will be administered at any suitable dosage. In one embodiment, the pharmaceutical compositions is administered parentally, i.e., intra-arterially, intravenously, subcutaneously or intramuscularly. In yet a further embodiment, the pharmaceutical compositions are for intra-tumoral or in-utero administration. In another embodiment, the pharmaceutical compositions are administered intranasally, intravitreally, subretinally, intrathecally or via other local routes.

The pharmaceutical composition may further comprises one or more pharmaceutically acceptable excipients. An excipient is a substance included in a pharmaceutical composition for the purpose of long-term stabilization, bulking up solid formulations that contain potent active ingredients in small amounts (i.e. may function as “bulking agents”, “fillers”, or “diluents”), or in some cases to enhance delivery of the active ingredient in the final dosage form, such as facilitating drug absorption, reducing viscosity, or modifying (often increasing) solubility. Excipients may also play a role in facilitating/improving manufacturing, e.g. acting as an antiadherent, binder, coating, glidant, lubricant, preservative, sorbent, and/or vehicle (for liquid and gel formulations). The term excipient encompasses the terms “carrier” and “diluent”. Non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington—The Science and Practice of Pharmacy, 21st edition (Gennaro et al editors. Lippincott Williams & Wilkins Philadelphia), each of which is incorporated by reference in its entirety. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions. Excipients include, but are not limited to, binders, fillers, flow aids/glidants, disintegrants, lubricants, stabilizers, surfactants, and the like.

The compositions described herein may be administered to a subject. The terms “patient” and “subject” as used herein includes a human or a non-human subject. As used herein, the terms “treat”, “treatment”, “therapeutic” and the like includes ameliorating symptoms, reducing disease progression, improving prognosis and reducing recurrence.

In some embodiments, the lipid nanoparticles have long circulation lifetimes, which may be beneficial to access extrahepatic tissues. In some embodiments, the lipid nanoparticles have improved scalability. In some embodiments, the lipid nanoparticles improve the internalization of GNP (or other metal nanoparticles) to avoid immune response issues. In some embodiments, the lipid nanoparticles have improved ability to efficiently encapsulate drug cargo.

### Triggered Release of Loaded Agent(s) of Interest

Lipid nanoparticles can be designed to accumulate at a tumor microenvironment where they release encapsulated therapeutics only at target cells, such as cancerous cells. Moreover, the vascular permeability of lipid nanoparticles (≤200 nm), which is increased by a well-known enhanced permeability and retention (EPR) effect, provides enhanced accumulation at the tumor microenvironment, thereby minimizing the undesirable side effects of chemotherapy (Greish, 2010, Methods in Molecular Biology, vol. 624, pp. 25-37; Jhaveri and Torchilin, 25 Apr. 2014, Frontiers in Pharmacology, Review vol. 5, no. 77, pp 1-26).

Lipid nanoparticles with long-term stability (e.g., no or limited cargo leakage under normal physiological conditions) can be designed to provide a controlled, sustained release of the encapsulated cargos by a stimuli-activation approach at the tumor microenvironment. Such internal stimuli-responsive delivery systems are advantageous in that they can destabilize the lipid nanoparticles (i.e., by degrading their structural components (Simões et al., 2001, Biochimica et Biophysica Acta (BBA)—Biomembranes, vol. 1515, no. 1, pp. 23-37; Li et al., 2015, Asian Journal of Pharmaceutical Sciences, vol. 10, no. 2, pp. 81-98; Yatvin et al., 1978, Science, vol. 202, no. 4374, pp. 1290-3)) in response to changes in cellular pH, redox conditions, enzymes and/or temperature at a target site.

By contrast to internally-stimulated release systems, external stimuli-responsive systems (e.g., using magnetic, ultrasound, thermal, microwave, radiofrequency, or light stimuli) have the potential to release their encapsulated cargo at a target microenvironment independently of differences in cellular mechanisms/phenomena between target and non-target sites (Chander et al., 2021, Small, vol. 17, no. 21, p. 2008198; Mathiyazhakan et al., 2014, Colloids and surfaces. B, Biointerfaces, vol. 126, pp 569-574). Examples include mild, local hyper-thermic trigger processes (e.g., ThermoDox™ technology), as well as phototherapies and photodynamic therapies.

Laser-triggered drug release from lipid nanoparticles may ensure a sharp transition temperature change, i.e., by only focusing the laser beam at the target tissue (deep in the body) without damaging the surrounding non-cancerous cells. This form of light-triggered drug release takes advantage of a pulsed laser (e.g., having femto, pico, or nanosecond pulses) with a peak in the near-infrared (NIR) region and thus can deliver a stronger and more focussed amount of energy (e.g., a highly localized temperature rise for a few nanoseconds in a few nanometers without excessive tissue heating) compared to other light-triggered system to exclusively release cargo. In order achieve such an effect, the lipid nanoparticle formulation process is adapted by incorporating within them plasmonic nanoparticles. This enables an amplified, localized electromagnetic plasma field to be specifically focussed at the inside of the liposomal targets to cause drug leakage in response to the pulsed laser irradiation (Boulais et al., 2012, Nano Lett, vol. 12, no. 9, pp. 4763-9; Boulais and Meunier, “Basic mechanisms of the femtosecond laser interaction with a plasmonic nanostructure in water,” Proceedings of SPIE—The International Society for Optical Engineering, vol. 7925, DOI:10.1117/12.876193; Patskovsky et al., 2014, The Analyst, vol. 139, pp. 5247-5253). Due to the strong, abrupt response of plasmonic nanoparticles to the incident light, an on/off resonance irradiation with an optimized laser fluence, whether a single shot or multiple shots (e.g., by tuning pulse-to-pulse spatial overlap and exposure time), can foster a controlled localised energy absorption and release energy, enabling site-specific drug release at the tumor microenvironment without harming non-target cells (Pustovalov, 2005, Chemical Physics, vol. 308, pp. 103-108; Pustovalov et al., Laser Physics Letters, vol. 5, pp. 775-792).

The LNPs disclosed herein may be triggered by various stimuli to release the loaded agent(s) encapsulated therein, which provides more targeted release of the loaded agents to result in increased efficacy and/or reduced adverse effects. Common stimuli include, without limitation, light (electromagnetic radiation), magnetic fields, temperature, ultrasound, pH, redox, or biochemical stimuli (e.g. enzymatic, and the like).

In one aspect, the LNPs or compositions disclosed herein are administered to a subject and the microenvironment of the target tissue (e.g. a tumour) triggers the release of the loaded agent(s), e.g. an anti-cancer agent, theranostic agent or tumour imaging agent.

In another aspect, the LNPs or compositions disclosed herein are administered to a subject and an external stimuli is administered (e.g. at a bodily target site) to release the loaded agent. A specific non-limiting example of external stimuli-triggered release is provided in Examples 6-8. For example, but without limitation, visible/infrared light can be used to trigger the release of agents of interest from gold or silver containing LNPs, radiofrequency radiation can be used to trigger release of agents of interest from gold containing LNPs, and magnetic fields can be used to trigger release of agents of interest from LNP containing iron oxide, cobalt or nickel, or an alloy of gold and any of the foregoing magnetic metals. In some embodiments, the inorganic particle comprises iron oxide and the external stimuli is a magnetic field. In some embodiments, the inorganic particle comprises gold and the external stimuli is an electromagnetic radiation (e.g. NIR or UV). In some embodiments, the inorganic particle comprises gold or silver and the external stimuli is irradiation from a light source or by a laser. In some embodiments, the inorganic particle is a metal nanoparticle (e.g. gold or silver), and the stimuli is irradiation (optionally using a laser) at a wavelength that is in resonance or out of resonance with a plasmonic peak of the metal nanoparticle. In some embodiments, the laser is a continuous wave or is pulsed. In some embodiments, the laser is pulsed with a pulsed width in microsecond, nanosecond, picosecond or femtosecond. In some embodiments, the irradiation is a femtosecond laser having a wavelength in resonance or out of resonance with the metal nanoparticle, wherein the metal nanoparticle is plasmonic.

A series of non-limiting embodiments are defined below.
Embodiment 1. A lipid nanoparticle comprising:
a helper lipid and an ionizable lipid, wherein the ionizable lipid is present at between 2 mol % and 30 mol % relative to total lipid;
at least one lipid layer surrounding an interior core, the interior core having at least one aqueous portion;
an encapsulated inorganic particle; and
an agent of interest, wherein the agent of interest is a hydrophilic agent present in the at least one aqueous portion or is a lipophilic agent present in the at least one lipid layer.
Embodiment 2. The lipid nanoparticle of embodiment 1, wherein the agent of interest is hydrophilic and is present in the at least one aqueous portion.
Embodiment 3. The lipid nanoparticle of embodiment 2, wherein the agent of interest is precipitated in the at least one aqueous portion.
Embodiment 4. The lipid nanoparticle of embodiment 2 or 3, wherein the at least one aqueous portion is acidic, and wherein the agent of interest is a weak base.
Embodiment 5. The lipid nanoparticle of embodiment 2 or 3, wherein the at least one aqueous portion is basic, and wherein the agent of interest is a weak acid.
Embodiment 6. The lipid nanoparticle of embodiment 1, wherein the agent of interest is lipophilic and present in the at least one lipid layer.
Embodiment 7. The lipid nanoparticle of embodiment 1, wherein the agent of interest is a hydrophilic agent conjugated to lipid moiety by a cleavable linker, and is present in the at least one lipid layer.
Embodiment 8. The lipid nanoparticle of any one of embodiments 1 to 7, wherein the inorganic particle present in the core is a metal nanoparticle.
Embodiment 9. The lipid nanoparticle of any one of embodiments 1 to 8, wherein the inorganic particle comprises gold or iron.
Embodiment 10. The lipid nanoparticle of any one of embodiments 1 to 9, wherein the inorganic particle present in the core is a colloid.
Embodiment 11. The lipid nanoparticle of any one of embodiments 1 to 10, wherein the inorganic particle present in the core has a diameter of 1 to 20 nm.
Embodiment 12. The lipid nanoparticle of any one of embodiments 1 to 11, wherein the lipid nanoparticle has an average diameter of 50 to 200 nm.
Embodiment 13. The lipid nanoparticle of claim 12, wherein the lipid nanoparticle has an average diameter of 70 to 150 nm.
Embodiment 14. The lipid nanoparticle of any one of embodiments 1 to 13, wherein the at least one lipid layer is a bilayer (bilamellar) or is multi-lamellar.
Embodiment 15. The lipid nanoparticle of embodiment 14, wherein the helper lipid is a neutral lipid and wherein a lipid component of the nanoparticle further comprises at least one of cholesterol and a hydrophilic polymer-lipid conjugate.
Embodiment 16. The lipid nanoparticle of any one of embodiments 1 to 15, wherein the helper lipid is present at a concentration of at least 20 mol %, optionally at least 30 mol %, or optionally 40 mol %.
Embodiment 17. The lipid nanoparticle of any one of embodiments 1 to 16, wherein the ionizable lipid is present at between 5 mol % and 15 mol % relative to total lipid.
Embodiment 18. The lipid nanoparticle of any one of embodiments 1 to 17, wherein a pH gradient exists across the lipid layer.
Embodiment 19. The lipid nanoparticle of any one of embodiments 1 to 18, wherein the inorganic particle is negatively charged.
Embodiment 20. The lipid nanoparticle of any one of embodiments 1 to 19, wherein the inorganic particle comprises a negatively charged cap.
Embodiment 21. The lipid nanoparticle of any one of embodiments 1 to 20, wherein the ionizable lipid is cationic at physiological pH.
Embodiment 22. The lipid nanoparticle of any one of embodiments 1 to 21, wherein the inorganic particle is complexed with the ionizable lipid and wherein the inorganic particle is located at an intersection of a lamellae of the lipid layer.
Embodiment 23. A method for producing a lipid nanoparticle entrapping an inorganic particle and an agent of interest, the method comprising:
(i) combining in two separate streams a first preparation of lipids dissolved in a solvent and a second preparation of an aqueous solution of an inorganic particle to produce a combined stream, thereby forming in the combined stream a lipid nanoparticle encapsulating the inorganic particle;
(ii) introducing a loading medium to an external solution of the lipid nanoparticle thereby formed, the external solution comprising the solvent, and allowing the loading medium to become entrapped in the lipid nanoparticle, thereby producing a lipid nanoparticle comprising the inorganic particle and the entrapped loading medium in an internal compartment thereof, and
(iii) introducing the agent of interest to an external solution of the lipid nanoparticle comprising entrapped loading buffer and allowing the agent of interest to be actively loaded into the lipid nanoparticle in response to the entrapped loading medium, thereby producing the lipid nanoparticle entrapping an inorganic particle and the agent of interest.
Embodiment 24. The method of embodiment 23, wherein a pH of the aqueous solution of the inorganic particle of step (i) is less than 5.5.
Embodiment 25. The method of embodiment 23 or 24, wherein lipids dissolved in the first preparation comprise an ionizable lipid, optionally a cationic lipid.
Embodiment 26. The method of embodiment 25, wherein the ionizable, cationic lipid is an amino lipid and the pH of the aqueous medium is less than a pKa of the ionizable, cationic lipid so that the ionizable, cationic lipid is charged.
Embodiment 27. The method of any one of embodiments 23 to 26, wherein the first and second preparations are pumped and mixed in a “T” junction mixer.
Embodiment 28. The method of any one of embodiments 23 to 27, wherein the lipid nanoparticle external solution is exchanged with a solution having a pH that is greater than a pH of the loading buffer by at least one pH unit.
Embodiment 29. The method of embodiment 28, wherein the loading medium is added to the lipid nanoparticle before the external solution is exchanged.
Embodiment 30. The method of any one of embodiments 23 to 29, wherein the solvent in the first preparation is ethanol.
Embodiment 31. The method of any one of embodiments 23 to 30, wherein the lipids in the first preparation comprise a helper lipid and an ionizable lipid, wherein the ionizable lipid is present at between 2 mol % and 30 mol % relative to total lipid, optionally between 5 mol % and 15 mol % relative to total lipid.
Embodiment 32. The method of embodiment 31, wherein the helper lipid is present at a concentration of at least 20 mol %, optionally at least 30 mol %, optionally at least 40 mol %.
Embodiment 33. A method for producing a lipid nanoparticle comprising an inorganic particle core and a lipophilic agent of interest, the method comprising:
   combining in two separate streams a first preparation of lipids dissolved in a solvent and a second preparation of an aqueous solution of an inorganic particle to produce a combined stream, thereby forming in the combined stream a lipid nanoparticle encapsulating the inorganic particle core;
   wherein the lipids in the first preparation comprise a helper lipid, an ionizable lipid, and the lipophilic agent of interest;
   wherein the ionizable lipid is present at between 2 mol % and 30 mol % relative to total lipid, optionally between 5 mol % and 15 mol % relative to total lipid; and
   wherein the helper lipid is present at a concentration of at least 20 mol %, optionally at least 30 mol %, optionally at least 40 mol %.
Embodiment 34. The method of embodiment 33, wherein the agent of interest is a hydrophilic agent of interest conjugated to a lipid moiety.
Embodiment 35. The method of embodiment 34, wherein the lipid moiety is conjugated to the hydrophilic agent of interest through a cleavable linker.
Embodiment 36. The method of any one of embodiments 23 to 35, wherein the lipid nanoparticle produced by the method is as defined in any one of claims 1 to 22.
Embodiment 36. Use of the lipid nanoparticle of any one of any one of embodiments 1 to 22 for delivering the agent of interest to a subject, wherein the lipid nanoparticle is for administration to the subject followed by administration of a stimulus to a region of the subject, the stimulus causing the inorganic particle in the lipid nanoparticle to cause release of the agent of interest from the lipid nanoparticle, optionally wherein the stimulus is electromagnetic irradiation, optionally from a light source or laser.
Embodiment 37. The use of embodiment 36, wherein the agent of interest is a therapeutic agent that treats a disease or condition of the subject.
Embodiment 38. The use of embodiment 36, wherein the agent of interest is an imaging agent, and wherein the use further comprises imaging the region of the subject.
Embodiment 39. The use of embodiment 36, wherein the agent of interest is a prodrug comprising a lipophilic therapeutic agent conjugated to a lipid moiety through a cleavable linker, wherein the use further comprising causing cleavage of the cleavable linker, and wherein after the cleavage the therapeutic agent treats a disease or condition of the subject.
Embodiment 40. A lipid nanoparticle comprising:
an ionizable lipid content of between 2 mol % and 30 mol %;
at least one of a hydrophilic polymer-lipid conjugate and a sterol;
a helper lipid content of greater than 30 mol % to form a bilayer surrounding an aqueous portion; an inorganic particle present in the bilayer; and
a therapeutic agent and/or imaging agent present in the aqueous portion,
wherein the therapeutic agent and/or imaging agent is releasable from the lipid nanoparticle by an irradiation.
Embodiment 41. The lipid nanoparticle of embodiment 40, wherein the therapeutic agent or imaging agent is precipitated in the aqueous portion.
Embodiment 42. The lipid nanoparticle of embodiment 40 or 41, wherein the inorganic particle is a metal nanoparticle and comprises gold or iron.
Embodiment 43. The lipid nanoparticle of embodiment 42, wherein the metal nanoparticle present in the aqueous portion is a colloid.
Embodiment 44. The lipid nanoparticle of any one of embodiments 40 to 43, wherein the lipid nanoparticle has an average diameter of 50 to 200 nm.
Embodiment 45. The lipid nanoparticle of embodiment 44, wherein the lipid nanoparticle has an average diameter of 70 to 150 nm.
Embodiment 46. The lipid nanoparticle of embodiment 45, wherein the metal nanoparticle present in the bilayer has a diameter of 1 to 20 nm.
Embodiment 47. The lipid nanoparticle any one of embodiments 40 to 46, wherein the helper lipid is a phosphatidylcholine lipid.
Embodiment 48. The lipid nanoparticle of any one of embodiments 40 to 47, wherein the helper lipid is present at a concentration of at least 35 mol %.
Embodiment 49. The lipid nanoparticle of any one of embodiments 40 to 47, wherein the helper lipid is present at a concentration of at least 40 mol %.
Embodiment 50. The lipid nanoparticle of any one of embodiments 40 to 47, wherein the helper lipid is present at a concentration of at least 45 mol %.
Embodiment 51. The lipid nanoparticle of any one of embodiments 40 to 50, wherein the cationic lipid is present at between 5 mol % and 15 mol % relative to total lipid.
Embodiment 52. The lipid nanoparticle of any one of embodiments 40 to 51, wherein a pH gradient exists across the lipid layer.
Embodiment 53. The lipid nanoparticle of embodiment 52, wherein the aqueous core is acidic and a solution external to the lipid nanoparticle is basic.
Embodiment 54. The lipid nanoparticle of any one of embodiments 40 to 53, wherein the therapeutic agent is a weak base and is pH-gradient loadable.
Embodiment 55. The lipid nanoparticle of any one of embodiments 40 to 54, wherein the metal nanoparticle is negatively charged.
Embodiment 56. The lipid nanoparticle of any one of embodiments 40 to 55, wherein the metal nanoparticle comprises a negatively charged cap.
Embodiment 57. The lipid nanoparticle of any one of embodiments 40 to 56, wherein the ionizable lipid is cationic below physiological pH.
Embodiment 58. A method for producing the lipid nanoparticle of any one of embodiments 1 to 22 or 40 to 57 comprising an ethanol mixing method.
Embodiment 59. Use of the lipid nanoparticle of any one of embodiments 1 to 22 or 40 to 57 to treat a mammalian subject in need of a treatment comprising triggered release of the agent at a bodily target site.
Embodiment 60. Use of the lipid nanoparticle of embodiment 59, wherein the triggered release is caused by an irradiation from a light source or by a laser.
Embodiment 61. Use of the lipid nanoparticle of embodiment 60, wherein the irradiation has a wavelength that is in resonance or out of resonance with a plasmonic peak of the metal nanoparticle.
Embodiment 62. Use of the lipid nanoparticle of embodiment 61, wherein the triggered release is caused by the laser and is in resonance.
Embodiment 63. Use of the lipid nanoparticle of embodiment 61, wherein the laser is a continuous wave or is pulsed Embodiment 64. Use of the lipid nanoparticle of embodiment 61, wherein the laser is pulsed with a pulsed width in microsecond, nanosecond, picosecond or femtosecond.
Embodiment 65. Use of the lipid nanoparticle of embodiment 61, wherein the irradiation is a femtosecond laser having a wavelength in resonance or out of resonance with the metal nanoparticles, wherein the metal nanoparticle is plasmonic.
Embodiment 66. A method of medical treatment comprising administering the lipid nanoparticle of any one of embodiments 1 to 22 or 40 to 57 to a mammalian subject in need of such treatment; and subjecting the lipid nanoparticle to an irradiation to trigger release of the agent at a bodily target site.
Embodiment 67. The method of embodiment 66, wherein the release is caused by an irradiation from a light source or by a laser.
Embodiment 68. The method of embodiment 66, wherein the irradiation has a wavelength that is in resonance or out of resonance with a plasmonic peak of the metal nanoparticle.
Embodiment 69. The method of embodiment 66, wherein the triggered release is caused by the laser and is in resonance.
Embodiment 70. The method of embodiment 66, wherein the laser is a continuous wave or is pulsed Embodiment 71. The method of embodiment 66, wherein the laser is pulsed with a pulsed width in microsecond, nanosecond, picosecond or femtosecond.
Embodiment 72. The method of embodiment 66, wherein the irradiation is a femtosecond laser having a wavelength in resonance or out of resonance with the metal nanoparticle, and wherein the metal nanoparticle is plasmonic.

The examples below are intended to illustrate the preparation of specific lipid nanoparticle preparations and properties thereof but are in no way intended to limit the scope of the invention.

### EXAMPLES

### Materials

The lipids (1,2-distearoyl-sn-glycero-3-phosphocholine (DSPC), 1,2-distearoyl-sn-glycero-3-phosphoethanolamine-N-[methoxy-(polyethylene glycol)-2000](PEG-DSPE), and the ionizable cationic lipid 1,2-dioleoyl-3-dimethylammonium-propane (DODAP)) were purchased from Avanti Polar Lipids (Alabaster, AL). Cholesterol (Chol), sodium acetate, ammonium sulfate (AS) and doxorubicin hydrochloride were obtained from Sigma-Aldrich Canada Ltd. (Oakville, Ontario, Canada). Phosphate buffered saline was from GIBCO (Carlsbad, CA). Dialysis membranes (molecular weight cutoff 12000-14000 Da) were from Spectrum Laboratories, Rancho Dominguez, CA). Amicon Ultracel centrifugal units (10 kDa MWCO) were from Millipore (Billercia, MA). Tannic acid stabilized negatively charged monodispersed spherical gold nanoparticles (5 nm diameter, particle concentration 5.5×10¹³ particles/ml) were provided by Ted Pella, Inc. (Redding, CA, USA) in the form of aqueous dispersions. The anion exchange spin columns (Vivapure D Mini H) were obtained from Sartorius Stedim Biotech, Aubagne, France. The QuantiFluo™ fluorimetric ammonia assay kit was obtained from BioAssay Systems (Hayward, CA). The Cholesterol E Total Cholesterol assay kit was provided by Wako Diagnostics (Richmond, VA).

### Methods

### Synthesis of Gold/Lipid Hybrid Nanoparticle Systems.

LNP-GNPs were prepared by a variation of the ethanol mixing method using a T-junction (Hirota et al., Biotechniques 1999, 27 (2), 286-90; incorporated herein by reference). Briefly, lipid nanoparticle-gold nanoparticle particles (LNP-GNPs) were formulated by mixing appropriate volumes of lipid stock solutions in ethanol buffer with an aqueous phase containing gold nanoparticles (GNPs) employing a T-tube mixer. Lipids (DODAP, DSPC, Chol and PEG-DSPE) were solubilized at a molar ratio of 10/49/40/1 to a final lipid concentration of 10 mg/ml in 100% ethanol. Aqueous gold colloids (with nominal particle diameters of 5 nm and particle concentration of 5.5 mg/ml as supplied by the manufacturer) were concentrated using centrifugal concentrators, and an appropriate volume of concentrated dispersion was then dissolved in 25 mM sodium acetate buffer, pH 4.0 to achieve a desired level of GNP number density. Acidification of the aqueous media was necessary to render the cationic lipid fully protonated (positively charged) to promote association with the negatively charged tannic acid cap of GNP. Lipids dissolved in ethanol and aqueous dispersion of GNP were pumped by means of two syringe pumps (volumetric flow rate ratio of 3:1 (aqueous to ethanol), pump rates 15 and 5 ml/minute, respectively) and mixed in a “T”-junction where two syringes containing organic and aqueous streams were connected to a union connector ( 1/16″, 0.02 in thru hole, IDEX Health & Science Part #P-712). Lipids were combined with GNPs at varying gold/lipids (Au/L) ratios ranging from 1.1×10¹³ to 8.8×10¹³ particles/mol lipid. Following mixing, gold/lipid dispersions were either dialyzed against formulation buffer to remove ethanol or dialyzed against 1× phosphate buffered saline, pH 7.4 to remove the residual ethanol and neutralize the buffer. Upon completion of dialysis, the mean diameter size together with ζ-potentials of spontaneously formed LNP-GNPs were determined (Zetasizer Nano ZS, Malvern Instruments Inc., Westborough, MA). Lipid concentrations were determined by measuring total cholesterol using the enzymatic assay. GNP entrapment efficiencies were measured by quantifying colloidal gold by measuring absorbance at 520 nm (absorbance maximum for 5 nm spherical GNP) in samples collected before and after removal of unentrapped gold using anion exchange spin columns and comparing the respective Au/L ratios. The absorbance measurements were performed upon lysis of the LNP-GNP and release of the entrapped gold nanoparticles by 1% Triton X-100.

### Synthesis of GNP Remote-Loadable Gold/Lipid Nanoparticle Systems.

To achieve the LNP-GNP systems capable to remote-load and stably retain the drug cargo, the freshly made lipid/gold mix dispersed in acetate buffer containing 25% ethanol (prepared as described above) was spiked with concentrated solutions of ammonium sulfate (AS). Briefly, 1 ml of aqueous AS (typically 0.9, 1.35 and 1.8 M) was drop-wise added to the 2 ml of vortexed lipid/gold dispersion, the resulting mix then placed into dialysis bags and dialyzed against phosphate buffered saline to remove the ethanol, unentrapped AS and raise the pH to 7.4. This procedure yields LNP-GNPs that entrap AS in amounts sufficient enough to provide the uptake and stable retention of the externally added drug via active loading mechanism27. The percentage of AS entrapment was determined by measuring concentration of ammonium in samples collected before and after dialysis (i.e., prior to and after removal of unentrapped ammonium) using the fluorimetric ammonia/ammonium assay kit. The measurements were carried out in the presence of 1% Triton X-100 to lyse the LNP-GNPs and release their contents. Particle size and lipid concentration measurements were performed as described above.

Loading of Therapeutic Agent into LNP-GNP.

Prior to loading, the ammonium sulfate-containing LNP-GNP systems were concentrated to approximately 10 mg/ml lipid using centrifugal concentrators. Doxorubicin hydrochloride was dissolved in saline at 5 mg/ml and mixed with the LNP-GNP dispersion to give the desired drug/lipid (D/L) ratios. The samples were then incubated at 60° C. to provide optimal loading conditions. Unentrapped doxorubicin was removed by running the samples over Sephadex G-50 spin columns prior to detection of entrapped drug. Doxorubicin was assayed by fluorescence intensity (excitation and emission wavelengths 480 and 590 nm, respectively) with a Perkin-Elmer LS50 fluorimeter (Perkin-Elmer, Norwalk, CT), the value for 100% release was obtained by addition of isopropanol to a final concentration of 50% vol. Drug loading efficiencies were determined by quantitating both drug and lipid levels in samples obtained before and after separation of unentrapped drug from LNP-GNP encapsulated drug by size exclusion chromatography using Sephadex G-50 spin columns and comparing the respective drug/lipid ratios.

### Cryo-Transmission Electron Microscopy (Cryo-TEM) Analysis of LNP Morphology.

Hybrid LNP-GNPs systems were imaged with a FEI Tecnai G20 TEM (FEI, Hillsboro, OR) using the method previously described by Leung et al., J Phys Chem B 2015, 119 (28), 8698-706; Witzigmann et al., Adv Drug Deliv Rev 2020, 159, 344-363, which are incorporated herein by reference. Prior to imaging, samples were concentrated to approximately 20 mg/mL total lipid, and 3-5 μl aliquot of concentrated dispersion was transferred to a glow-discharged copper grid in a FEI Mark IV Vitrobot. The sample was then plunge-frozen into liquid ethane to generate vitreous ice. Frozen samples were stored in liquid nitrogen until imaged. The TEM was operated at 200 kV in low-dose mode, and images were obtained using a bottom-mount FEI high-resolution CCD camera (FEI, Hillsboro, OR) at a nominal under focus of 2-4 μm. Sample preparation and image acquisition were performed at the UBC Bioimaging Facility (Vancouver, BC).

### Measuring Hydrophobic Prodrug in LNP-GNP.

INT-D034 was quantified by ultra-high pressure liquid chromatography (UPLC) using a Waters® Acquity™ UPLC system equipped with a photodiode array detector (PDA); Empower™ data acquisition software version 3.0 was used (Waters, USA). Separations were performed using a Waters® Acquity™ BEH C18 column (1.7 μm, 2.1×100 mm) at a flow rate of 0.5 ml/min, with a linear gradient from 80/20 (% A/B) to 0/100 (% A/B). Mobile phase A consisted of water and mobile phase B consisted of methanol/acetonitrile (1:1, v/v). The method was run over 6 minutes with a column temperature of 55° C. and the analyte was measured by monitoring the PDA detector at a wavelength of 239 nm.

### Example 1. LNPs Containing High Levels of Helper Lipid can be Generated that Contain Inorganic Particles as Well as an Aqueous Portion(s)

This example shows that LNPs which contain inorganic particles having an interior having aqueous portion(s) can be prepared by a solvent mixing process. Such LNP morphology is observed when the ionizable lipid content is lower and the helper lipid content is higher than used in conventional formulations to formulate nucleic acids in LNPs. Previous work has largely used the lipid composition ionizable lipid/cholesterol/DSPC/PEG-lipid in the molar ratios 50/38.5/10/1.5 to encapsulate nucleic acids.

A lipid composition having 10 mol % ionizable lipid (DODAP/DSPC/cholesterol/PEG-lipid 10/49/40/1, mol/mol) was selected to demonstrate inorganic particle (gold nanoparticle (GNP)) loading. The lipid mixture dissolved in ethanol was rapidly mixed with an aqueous solution containing the negatively charged inorganic particle (GNP) using a T-tube mixer. The aqueous solution was buffered at pH 4 so that the ionizable lipid was protonated and thus positively charged. Following the mixing step, the ethanol was removed by dialysis and the external medium was exchanged for phosphate buffered saline, pH 7.4 except for the micrograph shown in Figures A and C, where only the ethanol was removed and the sample was kept at pH 4 by dialysis against 25 mM sodium acetate buffer.

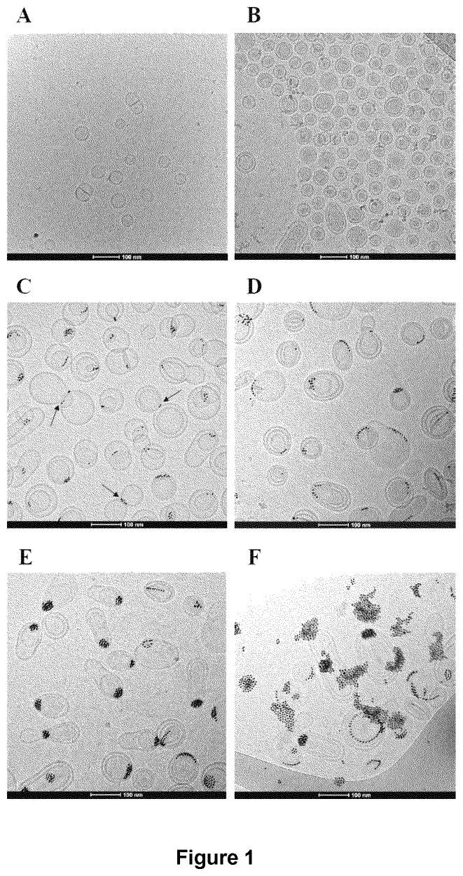

As shown in FIG. 1A, LNPs formed in the absence of GNPs at pH 4 (following ethanol removal by dialysis) are small (˜60 nm) unilamellar vesicles, some of them seen in a “hemifused” state. By contrast, adjustment of pH to 7.4 results in “fish eye” structures, with an inner lipid core within a lipid bilayer morphology (FIG. 1B). Such structures form due to high “helper” lipid content and can be accurately modeled under the assumption that the inner lipid core region of the interior corresponds to an oil core comprising the neutral form of the ionizable cationic lipid, surrounded by a monolayer of helper lipid and PEG lipid. The surrounding bilayer, on the other hand, may consist of, or consist essentially of, bilayer-forming helper lipid and PEG-lipid.

When GNPs were present in the formulation buffer, particles formed at pH 4 were either unilamellar or bi-lamellar vesicles with diameters of ˜100 nm. The GNP are either localized within bi-lamellar vesicles or are located at the interface between two unilamellar vesicles to produce “dumbbell” structures (FIG. 1C, indicated by arrows). After the pH is raised to pH 7.4, the particles observed are of comparable size but almost entirely bi- or oligolamellar (FIG. 1D-F). For Au/L ratios up to 6.6×10¹³ particles/mol lipid, at least a portion of the GNPs are localized in the interior of the vesicles formed. Further, the LNP advantageously also contain large aqueous compartments which provide potential for loading of small molecule drugs.

The cryo-TEM images shown in FIG. 1 D-E show that internalized GNPs are located at an apparent junction point between the inner and outer vesicles comprising the bilamellar and oligolamellar LNP. At least a portion of the ionizable cationic lipid may also be complexed with the negatively charged GNP in the same way that ionizable lipids are complexed to siRNA in LNPs encapsulating siRNA. Any excess ionizable lipid that is not associated with siRNA adopts a neutral oil form at pH 7.4 that segregates into hydrophobic domains. Thus, the external lipid bilayers of the hybrid LNP-GNP at pH 7.4 would be expected to contain little or no ionizable lipid and thus not exhibit a net positive surface charge. The ζ-potentials of the hybrid LNP-GNP systems prepared at various Au/L ratios were determined in dilute (×0.25) PBS as shown in Table 1. As noted, the negative ζ-potentials observed for LNP-GNP at pH 7.4 are similar to those obtained for DSPC/Chol systems determined under similar conditions in PBS, pH 7.4, consistent with the suggestion that there is little or no cationic lipid on the LNP-GNP surface.

TABLE 1
LNP-GNP: GNP content, size and surface charge characteristics.

| | | Mean diameter | |
|---|---|---|---|
| | | (Z-average | |
| Gold-to-lipid ratio (Au/L), | | means) by DLS, | Polydispersity |
| particles/μmol lipid | ζ-potential, mV | nm | index (PDI) |
| 2.2 × 10¹³ | −11.0 (0.45) | 88 (2.0) | 0.06 (0.01) |
| 3.3 × 10¹³ | −11.2 (0.43) | 87 (2.0) | 0.07 (0.01) |
| 4.4 × 10¹³ | −11.6 (0.40) | 87 (2.1) | 0.065 (0.01) |
| 6.6 × 10¹³ | −11.8 (0.35) | 89 (2.5) | 0.13 (0.02) |
| 8.8 × 10¹³ | −16.3 (0.47) | 114 (5.5) | 0.23 (0.03) |

Table 1. Sizes and ζ-potential values for LNP-GNPs prepared at various Au/L ratios. The LNP lipid composition was DODAP/DSPC/Chol/PEG-DSPE, 10/49/40/1 mol/mol and GNP encapsulation was performed as indicated under Methods. Measurements were taken on LNP produced after mixing at pH 4 and dialysis against PBS, pH 7.4. ζ-potential measurements were performed in dilute (×0.25) PBS. The standard deviation values from the repeat measurements are shown in brackets.

As noted above, cryo-TEM studies of formulations listed in Table 1 show no evidence of free GNP for LNP-GNP systems made with Au/L ratios up to 6.6×10¹³ particles/mol lipid, however, clumps of aggregated GNP are apparent for systems made at 8.8×10¹³ particles/mol lipid (FIG. 1B-D). Increasing the Au/L ratio from 2.2×10¹³ to 6.6×10¹³ particles/mol lipid did not affect the LNP-GNP's mean size and surface charge properties; however, the further increase to 8.8×10¹³ particles/mol resulted in growth in size and polydispersity as well as an apparent increase of the net negative surface charge. In the latter case, the data could reflect the co-existence of LNP-GNP with aggregates of (negatively charged) gold particles.

### Example 2: LNP Systems Containing High Levels of Bilayer Lipid can be Generated that Contain High Encapsulation of Gold Nanoparticles

The inventors next evaluated the GNP encapsulation efficiency achieved for the LNP-GNP systems at various Au/L ratios. Quantitation of GNP was performed by the surface plasmon resonance absorption assay, (Kreibig, U.; Vollmer, M., Theoretical Considerations. In Optical Properties of Metal Clusters, Springer Berlin Heidelberg: Berlin, Heidelberg, 1995; pp 13-201; incorporated herein by reference), which is a well-established technique suitable for determination of GNP in the presence of lipids solubilized by a detergent such as Triton X-100. For that purpose, a calibration plot was prepared ranging from 0 to 5.5×10¹³ particles/ml in presence of 1% Triton X-100, and the corresponding LNP GNP concentrations present in LNP-GNP samples prior to and following removal of external (unentrapped) gold were then calculated.

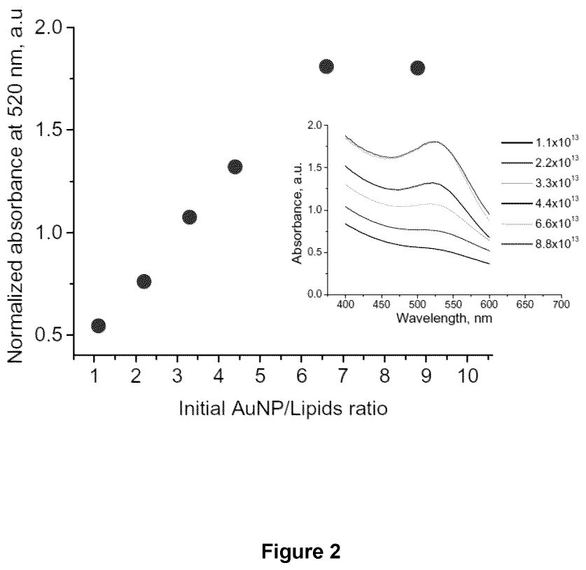

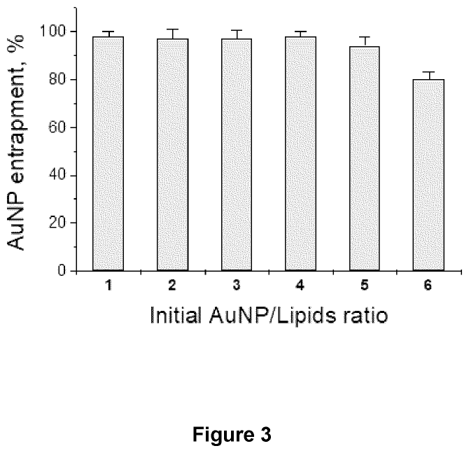

The UV-VIS absorption spectra for LNP-GNP systems at Au/L ratios from 1.1×10¹³ to 8.8×10¹³ particles/mol lipid) following removal of unentrapped gold (no detergent added) are shown in FIG. 2 (inset). All spectra exhibit a peak at ˜520 nm, a wavelength that is a fingerprint of colloidal gold nanoparticles. Quantitation of the gold content indicates trapping efficiencies approaching 100% for the systems prepared at Au/L ratios ranging from 1.1×10¹³ to 6.6×10¹³ particles/mol lipid, and increasing Au/L ratio 8.8×10¹³ particles/mol lipid results in ˜80% of gold being internalized (FIG. 3), in good agreement with results of Cryo-TEM studies (FIG. 1 B-D).

### Example 3: Therapeutic Agent can be Actively Loaded into the Aqueous Compartments of Hybrid LNP-GNP Systems

This example examines the ability to load drug into the LNPs in addition to GNP. The most robust procedure for drug encapsulation into LNP liposomal systems is to establish a pH gradient (inside acidic) and then load a weak base drug in response to the pH gradient. Over 50% of commonly used drugs detailed in the Merck Index are weak bases, making pH loading a generally applicable procedure. An effective method of generating the pH gradient is to entrap ammonium sulphate (AS) into the vesicles during formation and then remove exterior AS. The ammonium (NH₄⁺) can dissociate into NH₃⁺ H⁺, NH₃ can then readily permeate out, leaving an H⁺ behind and thus establishing a pH gradient.

TABLE 2
LNP after drug loading

| | Mean | | Mean | |
|---|---|---|---|---|
| AS concentration in | diameter, pre- | | diameter, | |
| formulation mixture, | loaded AuLNP | Polydispersity | drug-loaded | Polydispersity |
| mM | (nm) | index (PDI) | AuLNP (nm) | index (PDI) |
| 300 | 106 (1.8) | 0.06 (0.01) | 115 (2.4) | 0.08(0.01) |
| 450 | 114 (2.0) | 0.075 (0.015) | 123 (2.3) | 0.08 (0.015) |
| 600 | 127 (2.2) | 0.08 (0.01) | 135 (3.0) | 0.12 (0.02) |

Table 2. LNP-GNP sizes determined prior to and after loading (D/L 0.1 wt/wt) with doxorubicin. Measurements were taken after removal of ethanol and unentrapped AS (pre-loaded samples) and after removal of unentrapped drug (loaded samples) as described in Methods. Standard deviation values are shown in brackets.

There are various possible ways to introduce AS into the LNP during formation. One method involves incorporating high concentrations of AS into the aqueous medium containing the GNP during the mixing stage with the lipid in ethanol. However, aqueous dispersions of colloidal gold are sensitive to ionic strength. It was found that addition of an AS solution to a GNP dispersion resulted in precipitation. An alternative approach employed by the inventors herein was to add the AS after LNP formation at pH 4. It was reasoned that the change in LNP morphology as the pH is raised from pH 4 to pH 7.4 (see FIGS. 1A and B) may facilitate encapsulation and that the initial presence of 25% ethanol (v/v) may increase membrane permeability sufficiently to allow AS to permeate into the preformed LNP. The inventors therefore added concentrated (up to 1.8 M) aliquots of AS to the lipid dispersion containing GNP at pH 4 and then dialyzed against PBS. (See Methods for details).

The loading studies proceeded in two stages. The first stage was to determine how much AS could be encapsulated using the post-formulation addition of AS protocol where aliquots of concentrated AS were added dropwise to the GNP-containing (Au/L ratio 2.2×10¹³ particles/μmol) hybrid LNP at pH 4 to achieve final AS concentrations of 300 mM, 450 mM and 600 mM in the solution. This dispersion was dialyzed against PBS to remove residual ethanol, raise the pH and remove unentrapped AS. The resulting LNP GNP systems were then solubilized in the presence of detergent and assayed for ammonium and lipid content.

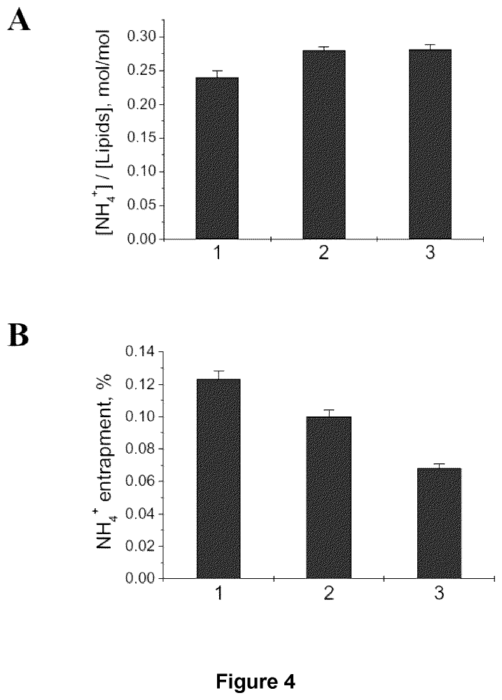

As shown in FIG. 4A, entrapped ammonium levels of 0.24 to 0.28 mol/mol lipid were achieved for initial AS concentrations at pH 4 of 300 mM to 450 mM. In terms of entrapment efficiency, these ammonium entrapment levels reflect a steady decline of trapping efficiency as the initial AS concentrations are increased (FIG. 4B). Importantly, the presence of AS during the dialysis step did not materially affect the LNP size or polydispersity, with only a slight size increase observed (Table 2).

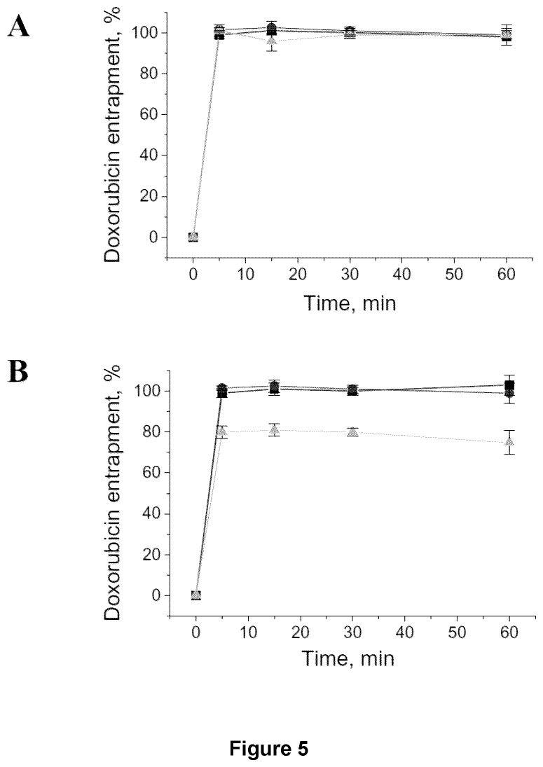

It was next demonstrated that the entrapped AS was sufficient to drive loading of an actively loadable therapeutic agent, in this case a weak base drug. The representative weak base drug chosen was the anticancer drug doxorubicin as doxorubicin can be loaded into liposomal LNP systems to such high levels that the drug precipitates inside the LNP, forming nanocrystals that can be readily imaged by cryo-TEM. Hybrid LNP-GNP samples (pH 7.4) containing AS were prepared as described above and an aliquot of doxorubicin solution was added and the formulation incubated at 60° C. using established doxorubicin loading protocols. The time course of doxorubicin uptake into hybrid LNP GNP AS systems prepared in the presence of 300, 450 and 600 mM AS was determined using an initial drug-to-lipid ratio of 0.1 (wt/wt) and is shown in FIGS. 5A-B. Doxorubicin trapping efficiencies approaching 100% were achieved within few minutes of incubation (FIG. 5A) for all samples.

Each doxorubicin molecule accumulated consumes a proton on arrival in the acidic interior, thus reducing the interior buffering capacity of the AS. If the drug-to-lipid ratio in the initial incubation medium is too high the buffering capacity will be exhausted and encapsulation efficiency reduced. We therefore investigated the effect of the initial drug-to-lipid ratios (0.05, 0.1 and 0.2, wt/wt) on encapsulation efficiency for hybrid LNP systems prepared in 450 mM AS at the pH 4 stage. As shown in FIG. 5B, varying the drug-to-lipid ratio between 0.05 and 0.1 resulted in effectively 100% encapsulation within 5 min of incubation. However, if the initial drug-to-lipid ratio was increased to 0.2 trapping efficiency decreased significantly. Drug loading resulted in a slight particle size growth as measured by DLS (Table 2).

The doxorubicin loading properties of the hybrid LNP-GNP-AS systems as shown in FIG. 5 are comparable to those reported for established formulations of liposomal doxorubicin such as Doxil™, but have the further advantage of encapsulating GNP. At high levels of encapsulation doxorubicin precipitates into fibrous-bundle nanocrystals that can be detected on cryo-TEM micrographs. Cryo-TEM studies of drug-loaded LNP-GNPs to detect such nanocrystals were therefore performed on an LNP-GNP (Au/L 2.2×10¹³ particles/μmol) formulation prepared in 450 mM AS (added at the pH 4 stage) and loaded at a drug-to-lipid ratio of 0.1 (wt/wt).

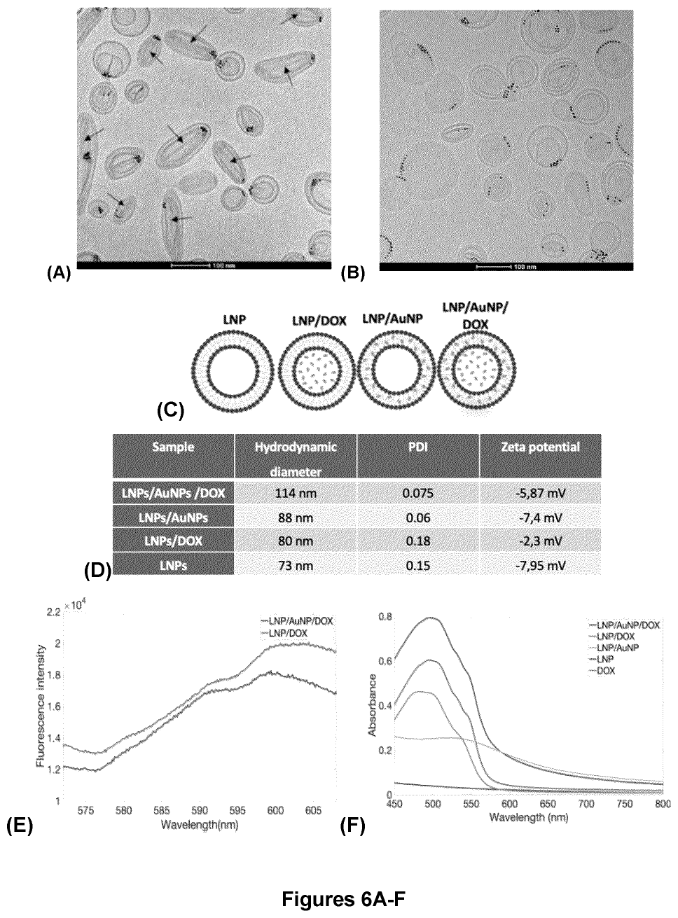

As shown in FIG. 6, structures reflecting precipitated drug can be observed in the hybrid LNP (indicated by arrows) which produce an elongated morphology of the LNP (compared to the more spherical LNP structures observed in the absence of drug, see FIG. 1B) were observed. This distortion may account for the slight size increase for drug-loaded LNP-GNPs as indicated by DLS. Overall, particles showing a distinctive drug precipitate formation represent a characteristic “coffee bean” appearance observed earlier for various liposomal doxorubicin formulations. Advantageously, the data shows the presence of both gold and therapeutic agent in the same lipid nanoparticle.

### Example 4: Hydrophobic Prodrugs can be Incorporated into Hybrid LNP-GNP Systems

This example examines the ability to load hydrophobic drugs into the LNPs that contain GNP. The lipid-like properties of lipophilic pro-drugs allow them to be easily loaded in LNP systems by simply mixing them with the lipid components. As an example, a dexamethasone prodrug, INT-D034, was incorporated into LNP-GNP systems. LNP-GNP systems were prepared as described earlier with modifications. INT-D034, ionizable or cationic lipid, DSPC, cholesterol and PEG-DSPE were mixed at a molar ratio of 10/10/43/36/1 in ethanol. Aqueous gold colloids (with nominal particle diameters of 5 nm and particle concentration of 5.5 mg/ml as supplied by the manufacturer) were concentrated using centrifugal concentrators, and an appropriate volume of concentrated dispersion was then dissolved in 25 mM sodium acetate buffer, pH 4.0 to achieve a desired level of GNP number density. Lipids dissolved in ethanol and aqueous dispersion of GNP were pumped by means of two syringe pumps (volumetric flow rate ratio of 3:1 (aqueous to ethanol), pump rates 15 and 5 ml/minute, respectively) and mixed in a “T”-junction where two syringes containing organic and aqueous streams were connected to a union connector. Formulations were dialyzed against PBS to remove residual ethanol. The physiochemical properties of the LNPs prepared as described above were subsequently characterized. Particle size was determined by dynamic light scattering using a Malvern Zetasizer Nano ZS (Malvern, UK) following buffer exchange into phosphate-buffered saline. Lipid concentrations were determined by measuring total cholesterol using the Cholesterol E enzymatic assay kit from Wako Chemicals USA (Richmond, VA). INT-D034 entrapment was determined using the UPLC.

Table 3 below shows that high encapsulation efficiencies for both GNP and prodrug are observed in LNP.

TABLE 3
LNP containing 5 nm GNP and INT-D034*

| Ionizable/ | % | GNP/μmol | Diameter | | % INT-D034 | % GNP |
|---|---|---|---|---|---|---|
| cationic lipid | INT-D034 | lipid | (nm, Z-avg) | PDI | entrapped | entrapped |
| DOTMA | 10 | 2.2 × 10¹³ | 82 | 0.143 | 91 | 99 |
| DODAP | 10 | 2.2 × 10¹³ | 78 | 0.037 | 89 | 100 |
| INT-A002** | 10 | 2.2 × 10¹³ | 73 | 0.097 | 89 | 100 |
| INT-A017*** | 10 | 2.2 × 10¹³ | 103 | 0.058 | 88 | 100 |
| DODAP | 0 | 2.2 × 10¹³ | 102 | 0.038 | N/A | 100 |
| DODAP | 0 | 2.2 × 10¹³ | 100 | 0.063 | N/A | 96 |
| DODAP | 0 | 0 | 83 | 0.107 | N/A | N/A |
| DODAP | 10 | 4.4 × 10¹³ | 79 | 0.063 | 81 | 98 |

(*) 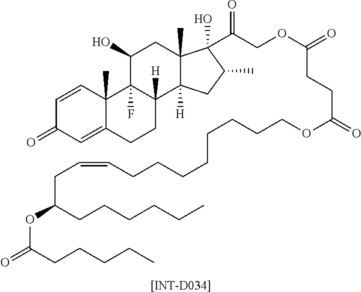 (**)3-(diethylamino)propyl (+)-syn-9,10-dilinoleoxystearate [INT-A002]
(***)3-[(±)-syn-9,10-(bislinoleoyloxy)octadecanoyloxy]-N,N-diethyl-N-methylpropan-1-aminium iodide [INT-A017]

### Example 5: Release of Agent of Interest (e.g. Drug) Using External Stimuli

Efficient agent of interest (e.g. drug) release from hybrid LNP containing GNP and agent of interest following electromagnetic irradiation in vitro—Hybrid LNP formulations containing GNP and doxorubicin (as exemplary agent of interest) are prepared as described above. Physical properties of LNP such as size/polydispersity and agent encapsulation efficiency are determined by dynamic light scattering and UPLC, respectively. Morphology of LNP is analyzed by cryo-TEM.

Various energy sources have been used to trigger drug delivery and release. GNP systems can engender triggered release as they can “explode” in response to high energy pulsed laser radiation (e.g., see: Letfullin, R. R.; Joenathan, C.; George, T. F.; Zharov, V. P., Laser-induced explosion of gold nanoparticles: potential role for nanophotothermolysis of cancer. Nanomedicine (Lond) 2006, 1 (4), 473-80), thereby disrupting LNP membranes or structure and promoting drug release. Desired wavelength of light source depends on the size of GNP (typically ranging from 200 nm to 1000 nm).

Breast cancer cell lines MCF-7 and MDA-MB-231 are incubated with various concentrations of LNP (10, 50, 100 μg/ml lipid) and subjected to light source. Cell viability is measured using commercially available MTS-based CellTiter 96™ AQᵤₑₒᵤₛ One Solution Cell Proliferation Assay (Promega) or the resazurin-based PrestoBlue™ assay (ThermoFisher). Reduced cell viability as compared to control cells without irradiation indicates triggered doxorubicin release.

Efficient agent of interest (e.g. drug) release from hybrid LNP containing GNP and agent of interest following electromagnetic irradiation in vivo—Hybrid LNP formulations containing GNP and doxorubicin (as exemplary agent of interest) are prepared as described above. Physical properties of LNP such as size/polydispersity and drug encapsulation efficiency are determined by dynamic light scattering and UPLC, respectively. Morphology of LNP is analyzed by cryo-TEM.

The effect of triggered doxorubicin release on anti-tumour efficacy of LNP systems is assessed in murine xenograft models. MCF-7 or MDA-MB-231 cells are implanted subcutaneously at the hind flank of Balb/c nude mice. Once tumours have reached a standard size (≈100 mm³), 8 mice per treatment group are injected i.v. with 3 escalating doses of hybrid LNP formulations. Electromagnetic irradiation using high energy pulsed lasers is applied at the tumour 12 to 24 hours post LNP injection. Desired wavelength of light source depends on the size of GNP (typically ranging from 200 nm to 1000 nm).

Anti-tumour efficacy is assessed by measurement of tumour size with callipers and data is plotted as median tumour volume as a function of time (tumour volume (mm³)=length×width²)/2). Suppression or reduction of tumour size in comparison to control animals without irradiation indicates successful triggered doxorubicin release in vivo. Repeat injection of LNP followed by electromagnetic irradiation is evaluated for comparison of anti-tumor effect.

### Example 6. Design and Synthesis of LNP Loaded with pH Gradient Loadable Agent (Dox) and Gold Nanoparticles (AuNP)

In order to produce LNPs that can encapsulate both hydrophobic negatively charged gold nanoparticles and a hydrophilic drug, such as Dox, the inventors used an LNP formulation of DODAP/DSPC/Chol/PEG-DSPE, 10/49/40/1 (mol/mol). The molar amounts of ionizable lipid (DODAP) and helper phospholipid (DSPC) were selected so that the LNP adopted a bilayer organization.

Dox was encapsulated in LNPs having a transmembrane pH gradient with an acidic interior. To facilitate this, the inventors developed a protocol to encapsulate ammonium sulfate (AS) into the aqueous core after incorporation of AuNPs into lipid nanoparticles, thus creating the pH gradient between the interior of the lipid nanoparticles and the exterior. The process to form the lipid nanoparticles follows several steps. In an initial step, the lipids are dissolved in ethanol. The resulting solution is subsequently mixed in an aqueous medium at pH 4, which contains the negatively charged AuNPs at a concentration of 3:1 aqueous medium to ethanol. This is the encapsulation step of the AuNPs. Subsequently, an aliquot of high concentration 450 mM ammonium sulfate was added. The mixture is finally dialyzed against physiological saline to increase the pH and remove the ethanol. These resulting lipid nanoparticles then show an outer layer composed almost entirely of Chol, DSPC and PEG.

The obtained LNPs were observed by Cryo-TEM imaging in FIGS. 6A and 6B. They show a spherical shape with a diameter close to 100 nm. Dox is found in the aqueous core of LNPs while AuNPs cluster in the outer layer as shown schematically in FIG. 6C. The physicochemical properties of the different LNPs prepared (with or without GNPs and Dox) are presented in the table shown in FIG. 6D. The LNPs all present close diameters in the different types of samples. However, a slight increase was observed when components like Dox or GNPs were encapsulated. The negative potential obtained is mainly related to the PEG layer on the outer surface of the LNPs. This PEG layer ensures the colloidal stability of the lipid nanoparticles in solution. Comparing different samples, the values vary because of the presence of Dox having a positive potential, and gold nanoparticles having a negative potential because of their surface coating.

The fluorescence of Dox is a property that is used for observation, and part of the emission spectrum is shown in FIG. 6E, with an excitation at the appropriate wavelength (505-555 nm) using an optical filter. Even encapsulated in LNPs containing AuNPs which are not fluorescent, it is possible to observe the fluorescence of Dox. The UV-vis absorbance spectra of the LNPs in FIG. 6F show the Dox absorption peak around 500 nm as well as the gold nanoparticles related peak around 540 nm, particularly visible on the LNP/AuNP sample. This suggests that it is possible to obtain a resonance effect on LNPs with a laser irradiating around 530 nm.

### Example 7: Release of Actively Loaded Agent (Dox) from Loaded LNP/AuNP/Dox Following Nanosecond Laser Irradiation

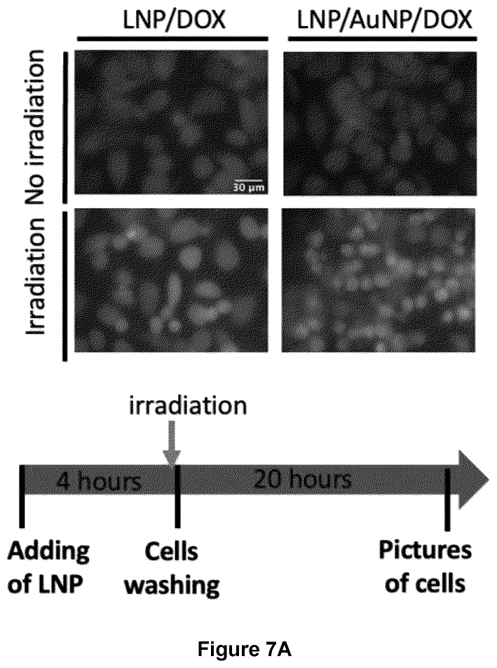

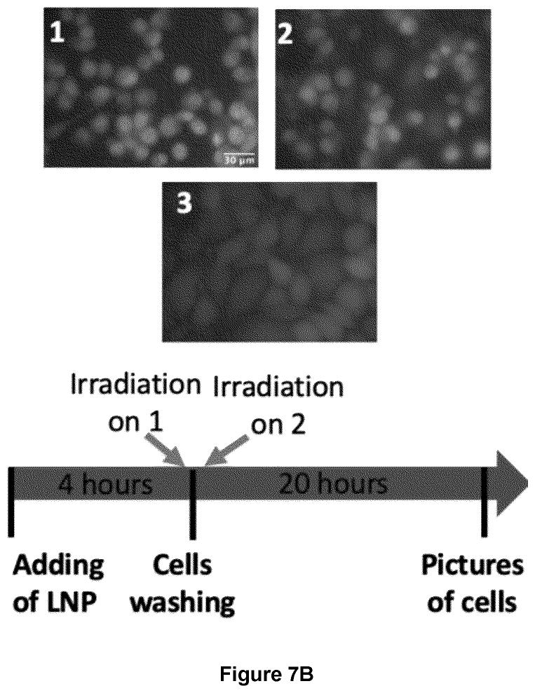

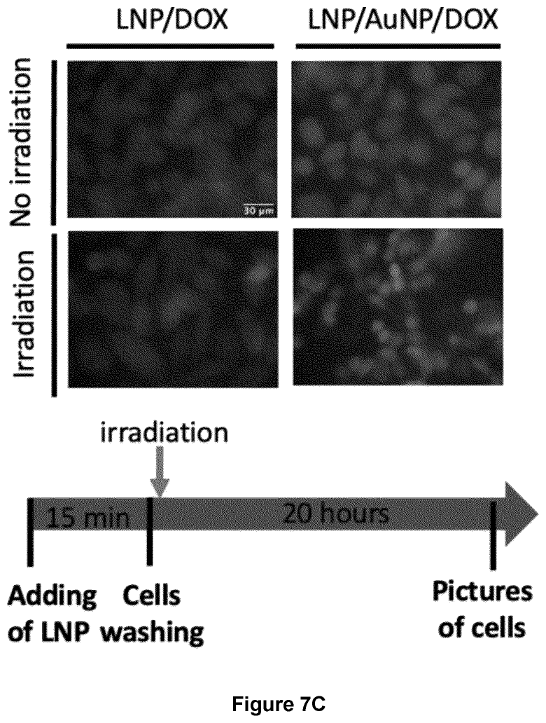

This experiment demonstrates laser irradiation to release Dox from LNP within MDA231 breast cancer cell lines. Several irradiation protocols were performed on the cells with an incubation of different samples of LNPs and controls. When these LNPs encounter the cells, they enter the cells through endocytosis. The agglomeration of LNPs in the endosomes can be seen in FIG. 7A, on the sample with LNP/AuNP/Dox without irradiation, by the presence of fluorescent red spots inside the cells. When Dox is released from the LNPs, it penetrates the nucleus and makes it strongly fluorescent as seen in FIG. 7A on the image with the irradiated sample when the LNPs contain AuNPs and Dox.

FIGS. 7A-C show representative fluorescent images using a nanosecond laser to trigger the Dox release from LNP/AuNP/Dox and compared to control experiments with LNP/Dox with and without irradiation and LNP/AuNP/Dox without irradiation. In each of these protocols, a washing step is performed to remove the LNPs present in solution, and thus reducing the fluorescence background. Experiments were performed with different incubation times of 4 hours and 15 minutes before irradiation and washing step as well as an irradiation before and after the washing step. For controls without irradiation, the LNP/Dox or the LNP/AuNP/Dox diluted in the culture medium to a concentration of 50 μg/mL (in LNPs) or 5 μg/mL in Dox were incubated during 4 hours or 15 mins according to the protocol. The influence of the time at which the irradiation is performed (in the presence or absence of LNPs in the surrounding medium) was studied by applying the protocol shown in FIG. 7B. After a minimum of 15 minutes of incubation time, time chosen to let the LNPs diffuse and start penetrating the cells, the samples were irradiated. The time at which the irradiation was performed did not affect the fluorescent intensity of the cells, and thus, the amount of Dox released that reached the nucleus. Indeed, as long as all the irradiations were performed in the same hour, it was verified that all the results were comparable. Only the incubation time, the time between the addition of LNPs and the washing of the cells, seems to be an important factor influencing the amount of Dox released. Fluorescent images were taken after a waiting time of 20 hours so that laser trigger released Dox could diffuse from the LNPs to the nuclei of the cells.

FIG. 7A shows that laser trigger Dox release occurs only in presence of both AuNP and irradiation. With or without irradiation, the images of the LNPs/Dox control samples are similar, and the laser does not release the drug, which remains encapsulated in the LNPs. A drug delivery result is only obtained in the case where there is a combination of irradiation with a presence of gold nanoparticles in the lipid nanoparticles. When both are present, it is possible to observe that the cell nuclei are highly fluorescent, a sign that Dox is released from the lipid nanoparticles and has reached the cell nucleus. Thus, during irradiation, the gold nanoparticles absorb the laser energy resulting in the release of the cargo included into the LNPs.

The second protocol of FIG. 7B shows that the moment at which the irradiation is performed (before or after washing). does not affect the quantity of Dox delivered to the nucleus of the cells. The irradiation only affects the LNPs which are already inside of the cells (entered via endocytosis) and not the LNPs which are still in the solution.

Moreover, FIG. 7C shows that even a small incubation time of 15 minutes of LNPs with cells is sufficient to let the LNPs enter the cells by endocytosis. The strong fluorescence of the nuclei, only visible when there is a combination of both the irradiation and the presence of gold nanoparticles in the outer layer of the lipid nanoparticles shows that Dox has been released. Note that for all these samples, all the cells are still alive since it is possible to see the fluorescence of Calcein AM, a marker of cell viability. However, the cells whose Dox could reach the nucleus after irradiation are in the process of cell death.

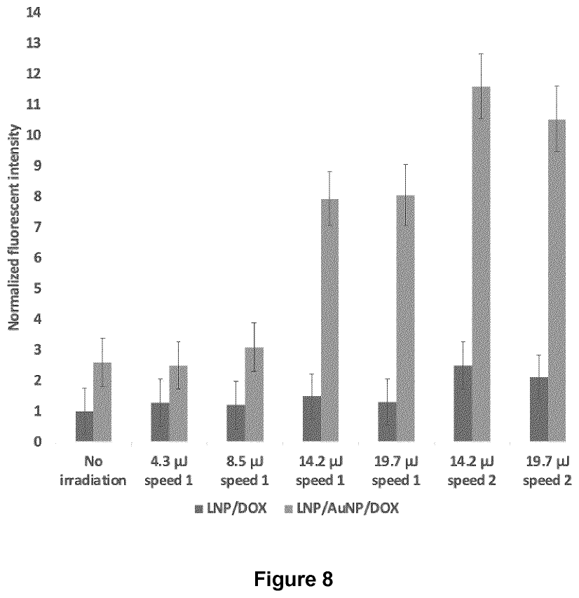

Other experiments were performed to quantify the drug delivered to the nucleus, thus evaluating the efficiency of the laser irradiation. To do so, it is considered that the measured fluorescence intensity of the nucleus is proportional to the Dox release. Measurements were performed on laser trigger Dox released from LNPs/AuNPs/Dox, as well as on Dox loaded LNPs and Dox loaded LNPs/AuNPs in medium with the same comparable concentration (5 μg/mL in Dox concentration). FIG. 8 shows the normalized fluorescent intensities to the condition of no-irradiation on LNP containing Dox for various irradiations and scanning parameters. Note that, even under irradiation, these normalized intensities stay relatively constant when no GNPs are present in the LNP, confirming that the GNPs play a necessary role in the Dox release. Two laser scanning conditions were used as denoted by speed 1 and 2, where each LNP/AuNP/Dox with no (speed 1) and some (speed 2) overlap between successive pulses. At speed 1, a threshold of irradiation energy at 14.2 μJ was found to effectively induce a Dox release from LNP/AuNP/Dox by a factor of 6 when compared to that of the irradiated LNP/Dox at this fluence. For the optimization of laser scan parameters, the speed 2 (50 μm/s and a 5 μm step between the scanned lines) was used in order to enable few laser pulses to overlap with each other in space, thereby maximizing the Dox release. When the speed is increased to 100 μm/s and 10 μm step (speed 2), the pulses do not overlap in the space, inducing a decrease of around 30% in the release percentage. In the best conditions, the irradiation of LNP/AuNP/Dox induced 11-fold increase of release comparing to that of the non-irradiated LNP/Dox sample.

### Example 8: Actively Loaded Agent (Dox) Release from LNP/AuNP/Dox Following Femtosecond Laser Irradiation

The inventors also explored the possibility of inducing the Dox release from LNP/AuNPs/Dox using a femtosecond laser (Spitfire, 800 nm, 55 fs, 1 kHz repetition rate, 35 μm spot diameter). When compared with the ns laser at 532 nm, the fs laser irradiates at a wavelength away from the Dox absorption peak, thus minimizing the unwanted photochemistry of the Dox. In addition, an irradiation in the weakly absorbing biological window (Barbora et al., 2021, PLoS One, vol. 16, no. 1, pp. e0245350-e0245350; Dabrowski et al., 2016, Coordination Chemistry Reviews vol. 325, pp. 67-101; Algorri et al., 2021, Cancers, vol. 13, no. 14, p. 3484) will lead to a much larger penetration depth for in vivo applications. While an irradiation at 800 nm is off-resonance of the plasmonic peak of a 5 nm AuNP, plasma mediated nanobubble formation may arise as it has been shown by Boulais et al. (Nano Lett, vol. 12, no. 9, pp. 4763-9, 2012), thus leading to the perforation of the LNP bilipid layer and the release of the Dox.

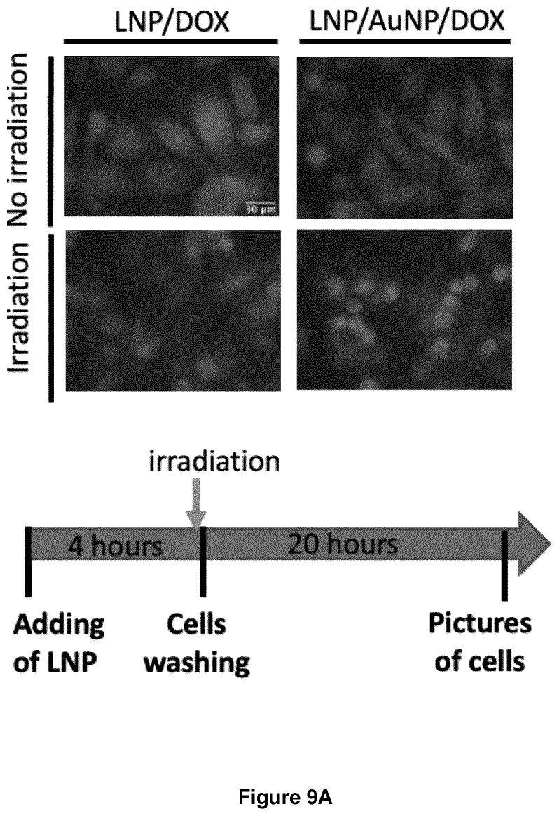

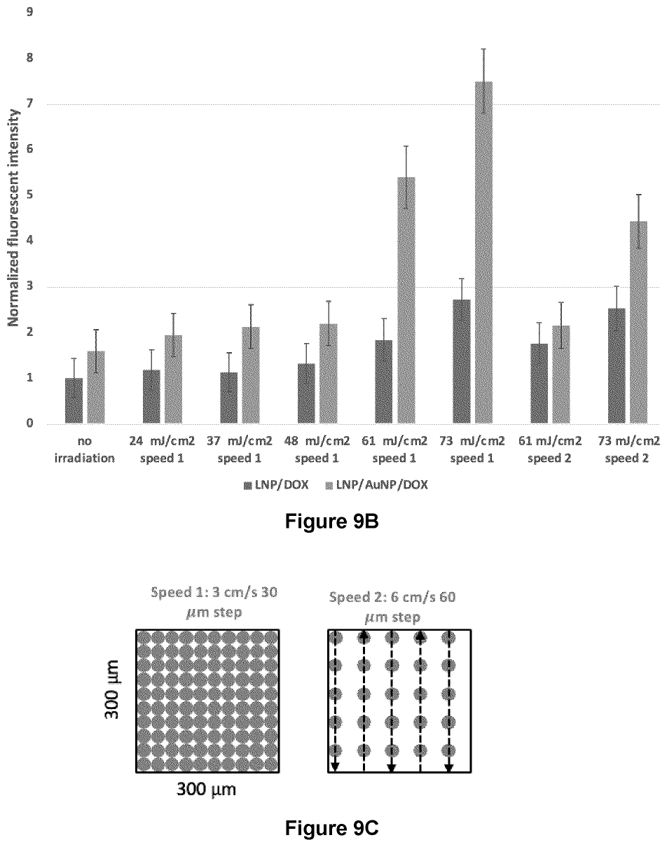

The irradiation protocol presented in FIG. 9A (bottom) and FIG. 9C is similar to the one used with the nanosecond laser and results show that indeed this laser can also trigger the Dox release from LNP/AuNP/Dox. At a fluence of 73 mJ/cm² and with laser pulses touching each other spatially (i.e., speed of 3 cm/s and 30 μm cm step between each scanned line), strong red fluorescence is observed in FIG. 9A. FIG. 9B shows the normalized fluorescent intensities to the condition of no-irradiation on LNP containing Dox for various irradiations and scanning parameters. As seen in FIG. 9B, a threshold is identified to induce the release of Dox at 61 mJ/cm² with a maximum obtained at 73 mJ/cm² when every pulse touches each other spatially. In those conditions, each nanoparticle receive a maximum of one pulse. In the best conditions, a 3×increase is observed between irradiated LNP/Dox and LNP/AuNP/Dox at a fluence of 73 mJ/cm², at the lowest speed. With this laser, a factor 7.5 between non irradiated LNP/Dox and irradiated LNP/AuNP/Dox in the best conditions was obtained.

The foregoing description is intended to illustrate embodiments of the invention and is in no way intended to limit the scope of the invention.

All documents cited in this disclosure are incorporated herein in their entirety. All priority documents are incorporated herein in their entirety. In the case of conflict between terms defined herein and defined in a cited document, the definitions in this disclosure will control.

1-46. (canceled)

47. A lipid nanoparticle comprising:
a helper lipid and an ionizable lipid, wherein the ionizable lipid is present at between 2 mol % and 30 mol % relative to total lipid;
at least one lipid layer surrounding an interior having at least one aqueous portion;
an encapsulated inorganic particle; and
an agent of interest, wherein the agent of interest is a hydrophilic agent present in the at least one aqueous portion or is a lipophilic agent present in the at least one lipid layer.

48. The lipid nanoparticle of claim 47, wherein:
(a) the agent of interest is hydrophilic and is present in the at least one aqueous portion; or
(b) the agent of interest is precipitated in the at least one aqueous portion;
optionally wherein:
the at least one aqueous portion is acidic, and the agent of interest is a weak base; or
the at least one aqueous portion is basic, and the agent of interest is a weak acid;
optionally wherein the agent of interest is pH-gradient loadable.

49. The lipid nanoparticle of claim 47, wherein:
(a) the agent of interest is lipophilic and present in the at least one lipid layer; or
(b) the agent of interest is a hydrophilic agent conjugated to lipid moiety by a cleavable linker, and is present in the at least one lipid layer.

50. The lipid nanoparticle of claim 47, wherein the agent of interest is a therapeutic agent and/or an imaging agent.

51. The lipid nanoparticle of claim 47, wherein the lipid nanoparticle has an average diameter of 50 to 200 nm, optionally 70 to 150 nm.

52. The lipid nanoparticle of claim 47, wherein the at least one lipid layer is bilamellar or is multi-lamellar,
optionally wherein the nanoparticle further comprises at least one of a sterol and a hydrophilic polymer-lipid conjugate, optionally wherein the sterol is cholesterol.

53. The lipid nanoparticle claim 47, wherein:
(a) the helper lipid is a neutral lipid, optionally a phosphatidylcholine lipid; and/or
(b) the helper lipid is present at a concentration of at least 20 mol %, optionally at least 30 mol %, optionally at least 35 mol %, optionally at least 40 mol %, or optionally at least 45 mol %; and/or
(c) the ionizable lipid is present at between 5 mol % and 15 mol % relative to total lipid; and/or
(d) a pH gradient exists across the at least one lipid layer surrounding the interior, optionally wherein the at least one lipid layer comprises a bilayer surrounding the interior, optionally wherein the aqueous portion is acidic and a solution external to the lipid nanoparticle is relatively basic; and/or
(e) the encapsulated inorganic particle is a colloid; and/or
(f) the encapsulated inorganic particle has a diameter of 1 to 20 nm.

54. The lipid nanoparticle of claim 47, wherein:
(a) the inorganic particle is negatively charged, optionally wherein the inorganic particle comprises a negatively charged cap; and/or
(b) the ionizable lipid is cationic at physiological pH or below physiological pH; and/or
(c) the inorganic particle is complexed with the ionizable lipid and wherein the inorganic particle is located at an intersection of a lamellae of the lipid layer.

55. The lipid nanoparticle of claim 47, wherein:
the at least one lipid layer comprises a bilayer surrounding the interior and the inorganic particle is in the bilayer surrounding the interior; and/or
the lipid nanoparticle further comprises an inner lipid core, and the inorganic particle is in the inner lipid core.

56. The lipid nanoparticle of claim 47, wherein the encapsulated inorganic particle is a metal nanoparticle, optionally wherein the encapsulated inorganic particle comprises gold, iron oxide, or hybrid gold-iron oxide.

57. The lipid nanoparticle of claim 47, wherein the agent of interest is releasable from the lipid nanoparticle using an external trigger, optionally an irradiation.

58. A method for producing a lipid nanoparticle entrapping an inorganic particle and an agent of interest, the method comprising:
(i) combining in two separate streams a first preparation of lipids dissolved in a solvent and a second preparation of an aqueous solution of an inorganic particle to produce a combined stream, thereby forming in the combined stream a lipid nanoparticle encapsulating the inorganic particle;
(ii) introducing a loading medium to an external solution of the lipid nanoparticle thereby formed, the external solution comprising the solvent, and allowing the loading medium to become entrapped in the lipid nanoparticle, thereby producing a lipid nanoparticle comprising the inorganic particle and the entrapped loading medium in an internal compartment thereof; and
(iii) introducing the agent of interest to an external solution of the lipid nanoparticle comprising entrapped loading buffer and allowing the agent of interest to be actively loaded into the lipid nanoparticle in response to the entrapped loading medium, thereby producing the lipid nanoparticle entrapping an inorganic particle and the agent of interest.

59. The method of claim 58, wherein:
(a) a pH of the aqueous solution of the inorganic particle of step (i) is less than 5.5; and/or
(b) lipids dissolved in the first preparation comprise an ionizable lipid, optionally a cationic lipid, optionally wherein the cationic lipid is an amino lipid and the pH of the aqueous medium is less than a pKa of the cationic lipid so that the cationic lipid is charged; and/or
(c) the first and second preparations are pumped and mixed in a “T” junction mixer; and/or
(d) the lipid nanoparticle external solution is exchanged with a solution having a pH that is greater than a pH of the loading buffer by at least one pH unit, optionally wherein the loading medium is added to the lipid nanoparticle before the external solution is exchanged; and/or
(e) the solvent in the first preparation is ethanol; and/or
(f) the lipids in the first preparation comprise a helper lipid and an ionizable lipid, wherein the ionizable lipid is present at between 2 mol % and 30 mol % relative to total lipid, optionally between 5 mol % and 15 mol % relative to total lipid, optionally wherein the helper lipid is present at a concentration of at least 20 mol %, optionally at least 30 mol %, optionally at least 35 mol %, optionally at least 40 mol %, or optionally at least 45 mol %; and/or
(g) the lipid nanoparticle produced by the method comprises:
a helper lipid and an ionizable lipid, wherein the ionizable lipid is present at between 2 mol % and 30 mol % relative to total lipid;
at least one lipid layer surrounding an interior having at least one aqueous portion;
an encapsulated inorganic particle; and
an agent of interest, wherein the agent of interest is a hydrophilic agent present in the at least one aqueous portion or is a lipophilic agent present in the at least one lipid layer.

60. A method for producing a lipid nanoparticle comprising an inorganic particle core and a lipophilic agent of interest, the method comprising:
combining in two separate streams a first preparation of lipids dissolved in a solvent and a second preparation of an aqueous solution of an inorganic particle to produce a combined stream, thereby forming in the combined stream a lipid nanoparticle encapsulating the inorganic particle core;
wherein the lipids in the first preparation comprise a helper lipid, an ionizable lipid, and the lipophilic agent of interest;
wherein the ionizable lipid is present at between 2 mol % and 30 mol % relative to total lipid, optionally between 5 mol % and 15 mol % relative to total lipid; and
wherein the helper lipid is present at a concentration of at least 20 mol %, optionally at least 30 mol %, optionally at least 35 mol %, optionally at least 40 mol %, or optionally at least 45 mol %.

61. The method of claim 60, wherein:
(a) the agent of interest is a hydrophilic agent of interest conjugated to a lipid moiety, optionally wherein the lipid moiety is conjugated to the hydrophilic agent of interest through a cleavable linker; and/or
(b) the lipid nanoparticle produced by the method comprises:
a helper lipid and an ionizable lipid, wherein the ionizable lipid is present at between 2 mol % and 30 mol % relative to total lipid;
at least one lipid layer surrounding an interior having at least one aqueous portion;
an encapsulated inorganic particle; and
an agent of interest, wherein the agent of interest is a hydrophilic agent present in the at least one aqueous portion or is a lipophilic agent present in the at least one lipid layer.

62. A method of delivering an agent of interest to a subject, comprising administering the lipid nanoparticle of claim 47 to the subject followed by administering a stimulus to a region of the subject, the stimulus causing the inorganic particle in the lipid nanoparticle to cause release of the agent of interest from the lipid nanoparticle, optionally wherein the stimulus is electromagnetic irradiation, optionally irradiation from a light source or by a laser.

63. The method of claim 62, wherein:
(a) the agent of interest is a therapeutic agent that treats a disease or condition of the subject; or
(b) the agent of interest is an imaging agent, and wherein the method further comprises imaging the region of the subject; or
(c) the agent of interest is a prodrug comprising a lipophilic therapeutic agent conjugated to a lipid moiety through a cleavable linker, wherein the method further comprises causing cleavage of the cleavable linker, and wherein after the cleavage the therapeutic agent treats a disease or condition of the subject.

64. A method for producing the lipid nanoparticle of claim 47 comprising an ethanol mixing method.

65. A method of medical treatment comprising administering the lipid nanoparticle of claim 47 to a mammalian subject in need of such treatment; and subjecting the lipid nanoparticle to an irradiation to trigger release of the agent at a bodily target site.

66. The method of claim 65, wherein the triggered release is caused by an irradiation from a light source or by a laser, optionally wherein the irradiation has a wavelength that is in resonance or out of resonance with a plasmonic peak of the metal nanoparticle, optionally wherein the triggered release is caused by the laser and is in resonance, optionally wherein the laser is a continuous wave or is pulsed, optionally wherein the laser is pulsed with a pulse width in microsecond, nanosecond, picosecond or femtosecond, optionally wherein the irradiation is a femtosecond laser having a wavelength in resonance or out of resonance with the metal nanoparticles, wherein the metal nanoparticle is plasmonic.
